(19) United States
(12) Patent Application Publication
(10) Pub. No.: US 2024/0335562 A1
(43) Pub. Date: Oct. 10, 2024

(54) SUSTAINABLE OCULAR CELL-MEDIATED INTRAOCULAR DELIVERY OF CELLULAR THERAPEUTICS FOR TREATMENT OF OCULAR DISEASES OR DISORDERS

(71) Applicant: Alpine Biotherapeutics Corporation, San Diego, CA (US)

(72) Inventors: Jiagang Zhao, San Diego, CA (US); Ying Wu, San Diego, CA (US)

(21) Appl. No.: 18/681,476

(22) PCT Filed: Aug. 4, 2022
(86) PCT No.: PCT/US2022/039437

§ 371 Date: Feb. 5, 2024

Related U.S. Application Data
(60) Provisional application No. 63/229,257, filed on Aug. 4, 2021.

(51) Int. Cl. A61K 48/00; A61K 38/17; C07K 14/71; C12N 15/86

(52) U.S. Cl. CPC A61K 48/005; A61K 38/179; A61K 48/0075; C07K 14/71; C12N 15/86; C12N 2740/15043

(57) ABSTRACT

Disclosed herein are novel compositions, engineered cells and cell lines, as well as related method of treating ocular diseases or conditions, such as neovascular age-related macular degeneration, diabetic retinopathy, glaucoma, corneal endothelial dystrophies and inherited retinal degenerative diseases, using sustainable ocular cell-mediated intraocular delivery of therapeutic agents or drugs.

### CROSS-REFERENCE TO RELATED APPLICATION(S)

This application claims priority to, and the benefit of, co-pending U.S. Provisional Application No. 63/229,257, filed Aug. 4, 2021. The disclosures of said provisional application are hereby incorporated by reference in their entirety.

### FIELD OF THE INVENTION

The present disclosure relates to methods of sustainable intraocular delivery of cellular therapeutics for treatment of ocular diseases or disorders.

### BACKGROUND OF THE INVENTION

The eye is a sensory organ that converts light signals into electric signals and then transmits them to the brain for imaging. Ocular degenerative conditions, such as age-related macular degeneration (AMD), inherited retinal degenerative diseases (IRDs), glaucoma, and corneal endothelial dystrophies (CED), are often associated with the irreversible deterioration of ocular tissue integrity and damage to specific cells. The irreversible cell loss can cause severe vision impairment or complete blindness in many patients. Currently, there are a few effective treatment methods with different drug delivery approaches available for the treatment of certain conditions, such as neovascular AMD.

Current treatment methods include the intravitreal (IVT) injection or administration of a drug to a subject. For example, in treating posterior segment ocular disease, given physical barriers to retinal access, including the blood-retina barrier, the treatment with large-molecule therapeutic agents, such as LUCENTIS and EYLEA, requires intravitreal injection (involving a needle stick in the subject's eye). Protein therapeutics, such as LUCENTIS, an anti-VEGF antigen-binding fragment (Fab), exhibit relatively short residence times in the vitreous humor following IVT injection, with elimination half-lives in the range of 6-10 days. As a result, frequent administration is required for a durable treatment effect. For example, the treatment of neovascular AMD with LUCENTIS requires administration to the subject every 4-8 weeks. Such frequent IVT administration carries a high treatment burden for patients, their caregivers, and physicians, and is associated with poor patient compliance with prescribed treatment regimens, resulting in less desirable results and treatment outcomes.

Additional treatment methods include gene therapy, which may involve placing copies of a gene into cells to capacitate them in producing the needed proteins to treat a condition. Gene therapy is a one-time treatment with the potential to generate long-lasting effects and eliminate the treatment burdens faced by patients receiving intravitreal injections by decreasing or eliminating the need for frequent injections. Progressive blindness is caused by inherited retinal diseases, such as retinitis pigmentosa (RP), which harbors mutations involved in over 60 genes as known so far. However, gene therapies under development only target a few caustic mutations for small populations of RP patients. For example, LUXTURNA, which has been approved as the first gene therapy for retinitis pigmentosa, can only treat about 2% of RP patients with a particular gene defect (RPE65). Patients with all other types of RP mutations (which represent about 98% of patients) still do not yet have an effective treatment available. Their vision becomes rapidly impaired and eventually will lead to severe visual disability. Currently, several other gene therapies are under development. Nevertheless, each of them will only address a low single-digit percentage of RP patients. Additionally, direct administration of recombinant viral particles carrying drug products may be associated with off-target and immunogenicity issues.

The transplantation of retinal pigment epithelial cells (RPE) is another method under clinical development for treating geographic atrophy AMD caused by progressive degeneration of RPE cells and photoreceptors. The mechanism of action of RPE cell transplantation mainly provides trophic protection for the remaining photoreceptors, with no cell regeneration involved. Thus, this method is unsuitable for the patients having advanced stages of degeneration, and who may only have a few residual healthy photoreceptors left in the macula.

Therefore, new compositions and methods are needed for the safe, long-acting and sustainable delivery of an effective amount of one or more agents (e.g., therapeutic agents) into the eye, and which have the potential to significantly improve the standard of care for the treatment and/or prevention of ocular diseases.

### SUMMARY OF THE INVENTION

Disclosed herein are new sustainable drug delivery approaches where ocular cells (e.g., ocular stem cells) are used for cell-mediated drug delivery based on the ocular cell therapy technologies developed by the present inventors. In particular, disclosed herein are compositions and methods that are useful for the ocular cell-mediated delivery (e.g., ocular stem cell-mediated delivery) and release (e.g., sustained release) of one or more therapeutic agents, drugs or biologics (e.g., a VEGF inhibitor) into the intraocular tissues of a subject, thereby providing a safe, effective, and sustainable release of such therapeutic agents, drugs or biologics to such subject's eye, and thereby treating various eye diseases or disorders affecting the subject. This new approach may be used to treat a broad spectrum of eye disease conditions, regardless of the underlying disease-causing genetic mutations.

In certain embodiments, the ocular cells disclosed herein comprise ocular stem cells. In certain embodiments, the ocular cells comprise ocular cell-fate further restricted precursor cells, such as retinal ganglion precursors, photoreceptor precursors, immature RPE cells, and corneal endothelial precursors, all as further described in Zhao, et al., Invest Ophthalmol Vis Sci. 2016 Dec. 1; 57(15):6878-6884, the contents of which are included by reference herein in their entirety. In certain embodiments, the ocular cell comprises an ocular cell-fate further restricted precursor cell. In certain embodiments, the ocular cell-fate further restricted precursor cell comprises a photoreceptor precursor cell. In certain embodiments, the ocular cell-fate further restricted precursor cell comprises a retinal ganglion precursor cell. In yet other embodiments, the ocular cell-fate further restricted precursor cell comprises a retinal pigmented epithelial (RPE) cell. In certain embodiments, the ocular cell-fate further restricted precursor cell comprises a corneal endothelial cell. In other embodiments, the ocular cell comprises the differentiated progenies of ocular stem cells.

Also disclosed herein are compositions and methods useful for the sustainable ocular cell-mediated intraocular delivery (e.g., ocular stem cell-mediated delivery) of biological drugs, such as inhibitors of vascular endothelial growth factor (VEGF) (e.g., aflibercept), a cytokine implicated in stimulating angiogenesis and targeted for intervention for the treatment of eye diseases or conditions such as, for example, neovascular age-related macular degeneration (nAMD), diabetic macular edema, diabetic retinopathy, other ocular disease caused by vascularization, inherited retinal diseases, and/or corneal endothelial dystrophy (CED).

Some embodiments of the present inventions include methods of sustainable ocular cell-mediated intraocular delivery (e.g., ocular stem cell-mediated delivery) of a biological drug into a subject's eye in need of treatment of an ocular disease or disorder with the drug. In certain aspects, such methods comprise a step of (a) preparing a drug expression construct that contains the biological drug coding sequence (e.g., SEQ ID NO: 1, encoding aflibercept) that is linked to a promoter, wherein the biological drug is linked to a leader sequence (e.g., a leader sequence comprising or selected from the group consisting of SEQ ID NO: 3, SEQ ID NO: 4, SEQ ID NO: 5, SEQ ID NO: 6, SEQ ID NO: 7 and/or SEQ ID NO: 8); (b) introducing the drug expression construct comprising or otherwise based upon a viral vector (e.g., an adeno-associated viral vector or lentiviral vector) or a mammalian plasmid expression vector into ocular cells (e.g., one or more ocular cells selected from the group consisting of ocular stem cells, photoreceptor precursors, retinal ganglion precursors, immature RPE cells and/or corneal endothelial cells) in vitro to form engineered ocular cells that can express the biological drug (e.g., aflibercept); (c) formulating engineered ocular cells that are able to produce or express the drug into a therapeutic composition; (d) injecting the therapeutic composition comprising the engineered ocular cells into the vitreous, the anterior chamber, the subretinal space, and/or the suprachoroidal space of the subject's eye; and (e) causing the production and secretion of the expressed drug (e.g., aflibercept) from the engineered ocular cells in a sustainable manner into the surrounding ocular tissues of the subject, and thereby providing a sustained therapeutic effect for treating the subject's ocular disease or disorder. In certain aspects, the drug expression construct comprises a sequence encoding aflibercept. For example, in certain embodiments, expression of the aflibercept coding sequence is driven by the human eukaryotic translation elongation factor 1 alpha1 short form promoter (EFS) in the mammalian gene expression lentiviral vector, and comprises a mammalian gene expression lentiviral vector (e.g., SEQ ID NO: 10).

Also disclosed herein are therapeutic compositions for treating ocular diseases or disorders (e.g., neovascular AMD) in a human subject in need thereof, such compositions comprising ocular cells that are introduced with a transgene expression vector in vitro, wherein the transgene expression vector comprises cis-regulatory and promoter sequences that control the expression of a transgene encoding a polypeptide of a VEGF inhibitor; and wherein the ocular cells comprising the transgene are formulated into a suspension of cells for intraocular administration to the human subject. In certain embodiments the transgene expression vector is an AAV or lentiviral vector. In certain embodiments, the ocular stem cells comprising the transgene are then cryopreserved for long-term storage and such cryopreserved ocular stem cells may be subsequently thawed and formulated into a suspension of cells for intraocular administration to the human subject. In certain aspects, the ocular cells comprise ocular stem cells. In certain embodiments, the ocular cells comprise an ocular cell-fate further restricted precursor cell (e.g., an ocular cell-fate further restricted precursor cell selected from the group consisting of a photoreceptor precursor cell, a retinal ganglion precursor cell, a retinal pigmented epithelial (RPE) cell, a corneal endothelial cell, and any combinations thereof). In certain embodiments, the ocular cell comprises a cell-fate restricted progeny of an ocular stem cell.

Certain embodiments of the present inventions are directed to ocular cells comprising the isolated mammalian ocular stem cells (OSCs) or primitive retinal stem cells (pRSCs), isolated mammalian retinal ganglion precursor cells (RGPCs), isolated mammalian photoreceptor precursor cell (PRPCs), isolated mammalian retinal pigment epithelial cells (RPECs), or isolated mammalian corneal endothelial cells (CECs). These ocular cells can be produced by in vitro methods, for example, as described in Zhao, et al., WO 2015/054526, U.S. Pat. No. 10,220,117 B2, and WO 2017/190136 A1, all of which are incorporated by reference in their entirety herein. For example, in certain embodiments, the ocular cells comprise isolated mammalian pRSCs and can be produced and isolated by: (a) culturing isolated pluripotent stem cells (PSCs) (e.g., embryonic stem cells (ESCs) or induced pluripotent stem cells (iPSCs)) from a mammal in a cell culture medium that is free of feeder cells, feeder-conditioned medium or serum so as to produce and grow a culture of the isolated PSCs; and (b) contacting the culture of the isolated PSCs so grown with one or more of an inhibitor for Wnt or TGF-β/BMP signaling so as to differentiate the isolated PSCs of (a) into primitive retinal stem cells, thereby producing isolated mammalian pRSCs, as described in Zhao, et al., WO 2015/054526. In certain embodiments, the ocular cells comprise isolated mammalian RGPCs, which can be produced and isolated by: (a) culturing isolated primitive retinal stem cells (pRSCs) from a mammal in a cell culture medium that is free of feeder cells, feeder-conditioned medium or serum so as to produce and grow a culture of the isolated pRSCs; and (b) contacting the culture of the isolated pRSCs so grown with one or more of an inhibitor of Wnt, Notch, or FGFR/VEGFR signaling so as to differentiate the isolated pRSCs into isolated RGPCs, thereby producing isolated mammalian RGPCs, as also described in Zhao, et al., WO 2015/054526.

In addition, in certain embodiments the mammalian retinal progenitor cells and corneal endothelial cells are directly isolated from donor tissues, for example, fetal retina and cornea donor tissues. These ocular cells can be used as a suitable vehicle and durable reservoir to deliver therapeutic agents or drugs to a diseased eye of a subject. Like pluripotent stem cells (PSCs) and other tissue stem cells, such as mesenchymal stem cells (MSCs), ocular stem/progenitor and precursor cells normally express low levels of immunogenicity markers. In addition, the eye is a well-known immune privileged organ. Recent data from a Phase 2b clinical trial of intravitreal administration of jCells (human fetal retinal progenitor cells) for RP treatment without immunosuppression drug also indicates the safety of ocular cells for sustainable drug delivery (see, Kuppermann, B., “Intravitreal Injection of Allogeneic Human Retinal Progenitor Cells (Jcell) for Treatment of Retinitis Pigmentosa,” The Retina Society). Accordingly, in certain aspects, the compositions and methods disclosed herein are characterized by their low immunogenicity and, for example, may be administered to a patient without immunosuppression and/or the administration of or pre-treatment with one or more immunomodulating agents.

In certain embodiments, the biological drug is or comprises a protein (e.g., a protein encoded by the amino acid sequence comprising SEQ ID NO: 1, or the nucleic acid sequence comprising SEQ ID NO: 2). In certain embodiments, the biological drug is or comprises RNA. In yet other embodiments, the biological drug is or comprises a complex of multiple moieties suitable for ocular cell-mediated delivery. In certain embodiments, the biological drug is or comprises any combination of the foregoing.

In certain embodiments, the inventions disclosed herein are generally directed to therapeutic compositions and related methods for treating an ocular disease or disorder in a subject (e.g., a human subject) in need thereof. In certain embodiments, such methods comprise a step of contacting one or more isolated ocular cells (e.g., ocular stem cells) with a transgene expression vector in vitro, wherein the transgene expression vector comprises cis-regulatory and promoter sequences that control the expression of a transgene encoding a polypeptide of a biological drug (e.g., a VEGF inhibitor); wherein the ocular stem cells comprising the transgene are formulated into a suspension of cells for intraocular administration to the human subject. In certain embodiments, expression of a coding sequence (e.g., the aflibercept coding sequence) may be driven by the human eukaryotic translation elongation factor 1 alpha1 short form promoter (EFS) in the mammalian gene expression lentiviral vector. In certain embodiments, the mammalian gene expression lentiviral vector comprises SEQ ID NO: 10.

In some embodiments, the ocular cells comprise one or more immature corneal endothelial cells (CECs) or corneal endothelial precursor cells (CEPCs), which are derived from ocular stem cells. For example, as described in Zhao, et al., WO 2017/190136 A1, the immature CEC or CEPC induction was driven by small molecules and in a stepwise fashion of lineage specification. During the initial phase, PSC fate was restricted to the eye field-like state and became eye field stem cell (EFSC). In the second phase, PSC-derived EFSC was further directed toward either neural crest lineage or retinal lineage. The CECs were directly induced from ocular neural crest stem cells (NCSCs) by suppressing TGF-beta and ROCK signaling. As disclosed herein, the CECs or CEPCs can be engineered (e.g., transfected with one or more expression constructs, such as an expression construct encoded by the sequence comprising SEQ ID NO: 10) and used as a vehicle to deliver the expressed biological drug (e.g., aflibercept) to the anterior chamber of the subject's eye for the treatment of corneal diseases.

Also disclosed herein are engineered ocular cell lines, wherein isolated cells of such engineered ocular cell line endogenously express a transgene encoding a polypeptide (e.g., a polypeptide encoding a VEGF inhibitor, such as aflibercept), and wherein the cells comprise an edited genome that results in the endogenous expression of the transgene compared to a control cell line. In certain embodiments, the polypeptide comprises SEQ ID NO: 1. In certain embodiments, the ocular cell line comprises ocular stem cells. In yet other embodiments, the ocular cell line comprises cell-fate further restricted precursors of ocular stem cells (e.g., ocular cell-fate further restricted precursors of ocular stem cells selected from the group consisting of photoreceptor precursor cells, retinal ganglion precursor cells, retinal pigmented epithelial (RPE) cells, corneal endothelial cells, and combinations thereof). In certain embodiments, the inventions disclosed herein are directed to methods of treating an ocular disease or disorder in a human subject (e.g., neovascular AMD) in need thereof, comprising administering one or more of engineered ocular cells disclosed herein to a subject (e.g., a human subject).

Some embodiments include the ocular stem cell-mediated sustainable intraocular drug delivery approach that can provide safe, effective, and sustainable release of therapeutic agents in a patient's eye for treating various eye diseases or disorders. This new approach may be used to treat a broad spectrum of eye diseases and conditions, regardless of the underlying disease-causing genetic mutation(s).

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee. The drawing illustrates a non-limiting embodiment of the technology. For clarity and ease of illustration, the drawings are not made to scale and, in some instances, various aspects may be shown exaggerated or enlarged to facilitate an understanding of particular embodiments.

FIG. 1 schematically depicts a diagram of the ocular cell-gene therapy methods disclosed herein for the treatment of ocular diseases or disorders by ocular cell-mediated delivery of a sustainably expressed biological drug. As depicted, ocular-fate restricted cells can be engineered in vitro to enable them to produce drug molecules with therapeutic effects, such as Aflibercept to treat neovascular ocular diseases or disorders. These engineered ocular cells are formulated to a therapeutic composition, which can then be administered (e.g., injected) into the vitreous (1), the anterior chamber (2), the subretinal space (3), or the suprachoroidal space (4) of a subject's diseased eye. The engineered ocular cells injected to the eye continuously produce and release the drug molecules to the surrounding ocular tissues of an injection site of the eye.

FIG. 2 depicts HEK293 cells that were transfected with the Lenti-Aflibercept expression construct (SEQ ID NO: 10) and showed the expression of a mCherry reporter at 48 hrs.

FIG. 3 depicts free soluble VEGF molecules in the culture supernatants of HEK293 cells and ocular stem cells (ABC101) transfected and transduced respectively either with or without aflibercept expression constructs. Compared with the controls, such as HEK293 cells transfected with Lipofectamine alone, ABC101 cells alone or ABC101 cells transduced with the virus of a reporter construct LV-EGFP-mCherry, VEGF was significantly reduced in the medium supernatants of HEK293 and ABC101 culture that expressed and secreted Aflibercept.

### DETAILED DESCRIPTION OF THE INVENTION

### General

The present inventions generally relate to compositions and related methods that comprise isolated ocular cells that have been engineered to endogenously express a transgene encoding a polypeptide (e.g., aflibercept). In certain embodiments, such ocular cells comprise isolated ocular stem cells (OSCs) that have been engineered to endogenously express a transgene encoding a polypeptide (e.g., aflibercept). In certain embodiments, such ocular cells comprise isolated retinal ganglion cells and/or retinal ganglion precursor cells that have been engineered to endogenously express a transgene encoding a polypeptide (e.g., aflibercept). In certain embodiments, such ocular cells comprise isolated retinal pigmented epithelial (RPE) cells that have been engineered to endogenously express a transgene encoding a polypeptide (e.g., aflibercept). In certain embodiments, such ocular cells comprise isolated corneal endothelial cells (CECs) that have been engineered to endogenously express a transgene encoding a polypeptide (e.g., aflibercept).

As used herein, the term “isolated OSCs” means that OSCs are substantially separated from contaminants (e.g., such as cells that are not OSCs). Likewise, the term “isolated RGCs” means that RGCs are substantially separated from contaminants (e.g., such as cells that are not RGCs). The term “isolated RPE or RPEs” means that RPE or RPEs are substantially separated from contaminants (e.g., such as cells that are not RPE or RPEs), the term “isolated CECs” means that CECs are substantially separated from contaminants (e.g., such as cells that are not CECs).

The terms “subject” or “patient” may be used interchangeably and refers to any living organism which can be administered any compositions derived from the present inventions (e.g., animals, typically mammalian animals, or humans). Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, and pigs) and experimental animal models (e.g., Drosophila, zebra fish, Xenopus, chick, mouse, rat, rabbit, guinea pig, and pig). In some embodiments a mammal is a human. A mammal can be any age or at any stage of development (e.g., an adult, teen, child, infant, or a mammal in utero). A mammal can be male or female. A mammal can be a pregnant female. In certain embodiments, a mammal can be an animal disease model. In some embodiments, the subject or patient is a human. In some embodiments, the subject or patient is an animal.

Sustainable Drug Delivery Approach where Ocular Stem Cells are Used for Cell-Mediated Drug Delivery Based on the Ocular Cell Therapy

Compositions and methods are disclosed herein for the sustainable intraocular delivery of an effective amount of one or more biological drugs via ocular cells (e.g., ocular stem cells). As used herein, the term “effective amount” refers to the amount of e.g., the cells of the invention and/or the therapeutic, biologic or drugs expressed by such cells that is required to retard, reduce or ameliorate at least one symptom of an eye-related disease or disorder (e.g., retinal degeneration). For example, an effective amount of any of the cells of the invention and/or the therapeutic, biologic or drugs expressed by such cells is the amount of effective to reduce or inhibit retinal degeneration of the cells of the invention. Thus, an effective amount is also the amount sufficient to prevent the development of an eye-related disease symptom, or to reduce a symptom or reduce the rate of symptom progression.

The biological drugs expressed by the cells of the present invention may be any therapeutic agent, biologic and/or drugs that can treat eye diseases or disorders, such as those inhibitors of vascular endothelial growth factor (VEGF), a cytokine implicated in stimulating angiogenesis and targeted for intervention for treating eye diseases or conditions such as neovascular age-related macular degeneration (nAMD), diabetic macular edema, diabetic retinopathy, or other ocular diseases caused by vascularization, inherited retinal diseases, and corneal endothelial dystrophy (CED).

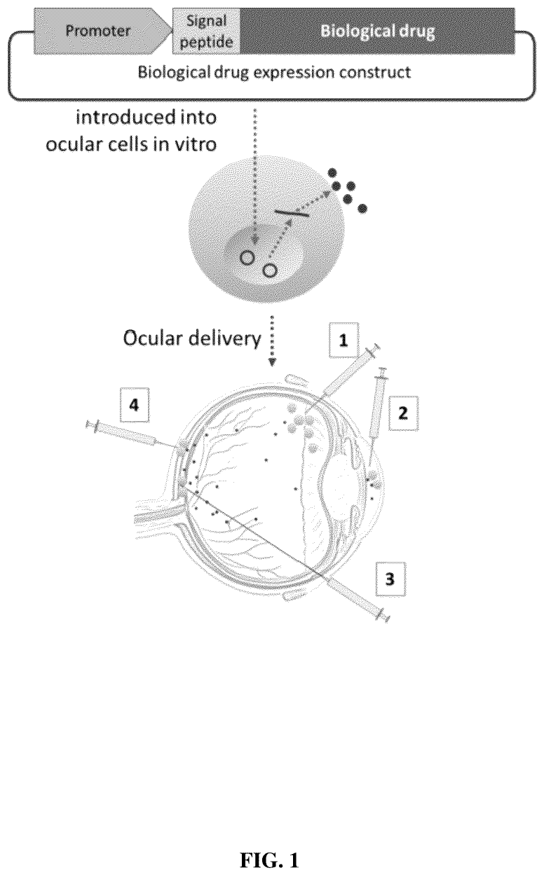

The method of the new sustainable intraocular delivery of cellular therapeutics via ocular stem cells for treatment of an ocular disease or a disorder include the following steps:
i. preparing a drug expression construct or a drug expression cassette that contains a therapeutic biological drug, which is linked to a promoter, and optionally a protein biomarker, wherein the biological drug is linked to a leader sequence;
ii. introducing the drug expression cassette of step (i) into ocular stem cells in vitro to produce engineered ocular cells;
iii. formulating the engineered ocular cells to produce a therapeutic composition;
iv. injecting the therapeutic composition comprising the engineered ocular cells of step (iii) into the vitreous (location 1 in FIG. 1), the anterior chamber (location 2 in FIG. 1), the subretinal space (location 3 in FIG. 1), or the suprachoroidal space (location 4 in FIG. 1) in need of treatment with the drug; and
v. the drug in the engineered ocular cells that are injected to the subject's eye in step (iv) being capable of continuously producing and releasing the drug molecules to the surrounding ocular tissues to provide therapeutic effect.

In certain aspects, the ocular cells disclosed herein are introduced or administered to the subject intraocularly (e.g., by injection into the subject's eye). As used herein, the terms “administering” and “introducing” are used interchangeably herein and refer to the placement of cells or compositions of the invention as disclosed herein into a subject by a method or route which results in at least partial localization of the cells or compositions at a desired site (e.g., intraocularly). The cells or compositions of the present invention can be administered by any appropriate route which results in an effective treatment in the subject. For example, in certain embodiments, the cells may be administered to subretinal space of the eye of the subject.

In one embodiment, the cells to be delivered may be combined with or contained on or in a matrix, e.g., a hydrogel, prior to delivery to the eye of the subject. In one embodiment, the hydrogel may comprise hyaluronic acid and methylcellulose or salts and derivatives thereof. Further, the hydrogel may comprise a hydrogel with a bioactive peptide. In one embodiment, the matrix may be a biocompatible polymer or mixture of biocompatible polymers which support cell viability and functionality of transplanted cells, artificial biomimetic matrix, bioactive scaffold derived from tissue or organ matrix, biosynthetic extracellular matrix based on collagen and N-isopropylacrylamide copolymers, or scaffolds modified with adhesion molecule, laminin, growth factor, morphogenetic factor, survival factor, extracellular matrix or fragment or derivative. The hydrogel may comprise about 0.5% sodium hyaluronate (1400-1800 kDa) and about 0.5% methylcellulose (100 kDa) or their equivalence in a balanced salt solution.

In some embodiments, a therapeutic composition can be prepared in the following ways: The ocular stem cells are transduced with a transgene expression vector such as AAV or lentiviral vector in vitro. The transgene expression vector comprises cis-regulatory and promoter sequences that control the expression of a transgene encoding a polypeptide of VEGF inhibitor such as aflibercept. For example, the DNA fragment comprised of the aflibercept coding sequence and the IL-2 leader sequence was de novo synthesized and subcloned into a lentiviral expression vector, which also carries a mCherry reporter expression cassette. The aflibercept expression construct is co-transfected with the envelope plasmid encoding VSV-G and packaging plasmids encoding Gag/Pol and Rev into HEK293T packaging cells. After 48 hours of incubation, the supernatant is collected and centrifuged to remove cell debris and then filtered. Lentiviral particles are subsequently concentrated with PEG. The ocular stem cells engineered with the transgene are then cryopreserved for long-term storage. The cryopreserved ocular stem cells are thawed and formulated into a suspension of cells prior to the intraocular administration step. The suspension of cells thus formed is a therapeutic composition for intraocular delivery to a subject's eye(s) via injection to treat eye diseases or disorders.

In this ocular cell-mediated drug delivery method, the ocular cells are a product of either the direct differentiation from pluripotent stem cells or the isolation and expansion of cells from donor tissues. The ocular cells can be derived from human iPSCs (for example, as disclosed in Zhao, et al., WO2015/054526 and WO 2017/190136A1, the contents of which are incorporated by reference in their entirety). The ocular cells include the isolated mammalian ocular stem cells (OSCs) or primitive retinal stem cells (pRSCs), isolated mammalian retinal ganglion precursor cells (RGPCs), isolated mammalian photoreceptor precursor cells (PRPCs), isolated mammalian immature retinal pigment epithelial cells (RPEs), and isolated mammalian immature corneal endothelial cells (CECs) which are produced by an in vitro method described in Zhao, et al., WO2015/054526 and WO 2017/190136A1. These ocular cells may be used as a suitable vehicle and durable reservoir to deliver therapeutic agents or products to the diseased eye. These authentic ocular progenitor and precursor cells do not need encapsulation to protect themselves from the host immune system.

In some embodiments, the ocular cells used for the delivery of the drug molecules are the isolated mammalian OSCs. In some embodiments, the ocular cells used for the delivery of the drug molecules are the isolated mammalian retinal ganglion precursor cells (RGPCs). In some embodiments, the ocular cells used for the delivery of the drug molecules are the isolated mammalian photoreceptor precursor cells (PRPCs). In some embodiments, the ocular cells used for the delivery of the drug molecules are the non-neural isolated mammalian immature retinal pigment epithelial cells (RPE). In some embodiments, the ocular cells used for the delivery of the drug molecules are the isolated mammalian immature corneal endothelial cells (CECs).

The therapeutic agent, biologic or drug that are expressed, produced or otherwise secreted by the ocular cells disclosed herein may be any therapeutic agents or products such as nucleic acids, proteins, and other drug moieties, that can treat eye diseases or disorders, for example, aflibercept, as exemplified in FIG. 1. Other drugs or cellular therapeutics may be used, such as brain-derived neurotrophic factor (BDNF), mitochondrial NADH-ubiquinone oxidoreductase chain 4 (MT-ND4) protein, and Retinoschisin (RS1), for the treatment of AMD, LHON, glaucoma, and inherited retinal diseases.

Many leader sequences may be used. Exemplary leader sequences include Interleukin-2 (IL2 leader) (e.g., SEQ ID NO: 3), IGFBP (e.g., SEQ ID NO: 4), VEGF-A (e.g., SEQ ID NO: 5), Vitronectin (e.g., SEQ ID NO: 6), Albumin (e.g., SEQ ID NO: 7), and/or Complement Factor H (e.g., SEQ ID NO: 8). In some embodiments, the leader sequence is IL2 signal peptide.

TABLE 1
Leader Sequences

| | | SEQ |
|---|---|---|
| ID | Sequence | ID NO: |
| IL2 | MYRMQLLSCIALILALVINS | 3 |
| IGFBP | MLPRVGCPALPLPPPPLLPLLLLLLGA | 4 |
| | SGGGGGARA | |
| VEGF-A | MNFLLSWVHWSLALLLYLHHAKWSQA | 5 |
| Vitronectin | MAPLRPLLILALLAWVALA | 6 |
| Albumin | MKWVTFISLLFLESSAYS | 7 |
| Complement | MRLLAKIICLMLWAICVA | 8 |
| Factor H | | |

In some embodiments, the promoter is a constitutively active promotor. In some embodiments, the expression vectors (e.g., plasmids, AAV, and lentiviral vectors which have been used for gene therapy in clinical settings) may be used as a promoter to transduce the authentic ocular stem cells. The vector contains a biological factor coding sequence lead by a signal peptide sequence. For example, an expression cassette of IL2 leader sequence fused to aflibercept ORF (open reading frame) has been constructed, as depicted in FIG. 1.

The drug molecules can be expressed in the engineered ocular stem cells, and the expressed drug molecules then can be released from or secreted by the engineered ocular stem cells. Once the engineered ocular stem cells containing the drugs are injected into a subject's eye, the transplanted cells not only can continuously release the drug molecules, such as aflibercept, that inhibit the overgrowth of blood vessels, but also provide trophic support or nutrition to rescue retinal visual cells that are degenerating in a subject's eye.

In some embodiments, the engineered ocular stem cells may be transplanted to the subretinal space of the macula and surrounding areas, where the transplanted ocular stem cells then integrate into the retinal structure and do three different things. First, such transplanted ocular stem cells release a drug, such as aflibercept, directly into the macular region to block the invasion of blood vessels. Second, such transplanted cells serve as a durable reservoir of trophic or nutritive factors to rescue the dying retinal cells in the area. Third, such transplanted cells can differentiate into new retinal pigment epithelium (RPE) and retinal visual cells and repair the visual circuitry necessary for sight.

In some embodiments, the sustainable delivery of the therapeutic agent or drug by the transplanted engineered ocular stem cells may be used to treat nAMD patients. Currently, the available treatment methods for nAMD include laser photocoagulation, photodynamic therapy with verteporfin, and the standard of care treatment with intravitreal injections with agents aimed at binding to and neutralizing VEGF. Exemplary anti-VEGF agents include Ranibizumab (a small anti-VEGF Fab protein which was affinity-improved and made in prokaryotic E. coli), Bevacizumab (a humanized monoclonal antibody (mAb) against VEGF produced in CHO cells), and aflibercept (a recombinant fusion protein consisting of VEGF-binding regions of the extracellular domains of the human VEGF-receptor fused to the Fc portion of human IgGi, which belongs to a class of molecules commonly known as “VEGF-Traps”). Each of these therapies have improved best-corrected visual acuity (BCVA) on average in naive nAMD patients. However, their effects appear limited in duration and patients usually receive frequent doses every 4 to 6 weeks on average.

To achieve a long-term release of an VEGF inhibitor without the burden of repeated injections, for example, a transgene of aflibercept is expressed under the control of a ubiquitous promoter such as EFlalpha core promoter sequence identified in Table 2 (SEQ ID NO: 9). A human IL2 signal peptide is placed at the N-terminus of aflibercept (FIG. 1). The viral expression vectors can be used to deliver the transgene into the PSC-derived ocular cells. These vectors may include replication-deficient recombinant adeno-associated viral vectors as well as lentiviral vectors. Plasmids or non-viral expression vectors may also be utilized. Since the anti-VEGF proteins are expressed in human ocular cells, it is expected that they will likely have full human post-translational modifications.

TABLE 2
Promotor Sequences

| | | SEQ |
|---|---|---|
| ID | Sequence | ID NO: |
| EF1 alpha | ggctccggtgcccgtcagtgggcagagcgcaca | 9 |
| | tcgcccacagtccccgagaagttggggggaggg | |
| | gtcggcaattgatccggtgcctagagaaggtgg | |
| | cgcggggtaaactgggaaagtgatgtcgtgtac | |
| | tggctccgcctttttcccgagggtgggggagaa | |
| | ccgtatataagtgcagtagtcgccgtgaacgtt | |
| | ctttttcgcaacgggtttgccgccagaacacag | |
| | g | |

Ocular Stem Cells from iPSCs

Cell therapy represents a tremendously promising approach by replenishing the injured tissues with healthy cells, regardless of underlying genetic or acquired cause. Substantial evidence suggests that ocular progenitor and precursor cells could be used as potential drug products to treat vision loss (see, Wang Y, et al., Cell Death Dis. 2020 Sep. 23; 11(9):793. doi: 10.1038/s41419-020-02955-3. PMID: 32968042; PMCID: PMC7511341.). Preclinical studies demonstrated that the transplantation of these cells into the eye could result in photoreceptor replacement and significant slowing of host photoreceptor loss. A subset of the progenitors developed into mature neurons, including presumptive photoreceptors expressing recoverin, rhodopsin, or cone opsin. Rescue of cells in the outer nuclear layer (ONL), along with widespread integration of donor cells into the inner retina of recipient mice, showed improved light-mediated behavior compared with the control animals (see, Semo M, et. al., Trans Vis Sci Tech. 2016; 5(4):6; and Wang Z, et. al., Med Sci Monit. 2020 Mar. 28; 26:e921184. Doi: 10.12659/MSM.921184. PMID: 32221273; PMCID: PMC7139196).

In addition, the cell therapy approach has been clinically validated for retinal and corneal care. In early phases of clinical trials, subjects treated with healthy fetal retinal progenitor cells (RPCs) or corneal endothelial cells (CECs) expanded from a donor tissue have experienced significant and durable improvements in visual acuity. The treatment of intravitreally delivered RPCs was well-tolerated, safe, and efficacious in halting and even reversing the vision loss among RP patients. Notably, no immunosuppression drug was used in the RPC cell therapy studies. It represents a significant advantage for progenitor cells over more matured cell products, which often require immune suppression. However, relying on fetal donor tissue causes concerns on the limitation of source material, scalability of manufacture, and ethical conflicts. Thus, there is a significant clinical need for an alternative authentic ocular cell source.

During the early stages of embryonic head development, the eye primordium or eye field is formed under the influence of WNT and BMP signaling gradients. The eye field cells are gradually restricted to different ocular lineages and cell fates. Remarkably, this eye development process can be mimicked in vitro by the differentiation of human pluripotent stem cells (ESCs or iPSCs) through a targeted and stepwise process. The present inventors have previously developed a highly efficient, small-molecule-based method to induce ocular stem cells from iPSCs in vitro under a defined set of culture conditions, as further described in Zhao, et al., WO 2015/054526, the contents of which are incorporated by reference in their entirety).

In some embodiments, ocular stem cells (OSCs) (or eye field stem cells (EFSCs), or primitive retinal stem cells (pRSCs)) are produced in vitro by the method described in Zhao, et al., WO2015/054526, which comprises (1) culturing isolated pluripotent stem cells (PSCs) from a mammal in a cell culture medium that is free of feeder cells, feeder-conditioned medium or serum so as to produce and grow a culture of the isolated PSCs; and (2) contacting the culture of the isolated PSCs with one or more of an inhibitor for Wnt or TGF-β/BMP signaling so as to differentiate the isolated PSCs into OSCs, thereby producing isolated mammalian OSCs. In some embodiment, one or more of an inhibitor for Wnt or TGFβ/BMP signaling is a combination of inhibitors for Wnt and TGF-β/BMP signaling. The culture of isolated PSCs may be an adherent culture. In some embodiment, the culture of the isolated PSCs is a monolayer culture. In some embodiment, the culture of isolated PSCs is grown to near confluence before contacting with one or more of an inhibitor for Wnt or TGFβ/BMP signaling or a combination of inhibitors for Wnt and TGF-β/BMP signaling. In some embodiments, an inhibitor for Wnt or TGFβ/BMP signaling is a small molecule inhibitor. The induced pluripotent stem cells (iPSCs) from a mammal may be similarly treated in place of isolated PSCs to produce isolated mammalian OSCs. In some embodiments, the isolated mammalian OSCs are directed to differentiate toward specific retinal cell fates in vitro using small molecule inducers of differentiation. In some embodiments, the specific retinal cell fates include neuroretinal cells and non-neuronal cells. In some embodiments, the neuroretinal cells include retinal ganglion precursor cells (RGPCs) and photoreceptor precursor cells (PRPCs). In some embodiment, the non-neuronal cells include retinal pigment epithelial cells (RPECs).

The isolated mammalian retinal ganglion precursor cells (RGPCs) may be produced by an in vitro method comprising: (a) culturing isolated primitive retinal stem cells (pRSCs) from a mammal in a cell culture medium that is free of feeder cells, feeder-conditioned medium or serum so as to produce and grow a culture of the isolated pRSCs; and (b) contacting the culture of the isolated pRSCs so grown with one or more of an inhibitor of Wnt, Notch, or FGFR/VEGFR signaling so as to differentiate the isolated pRSCs into isolated mammalian RGPCs, thereby producing isolated mammalian RGPCs.

The isolated mammalian photoreceptor precursors from isolated mammalian pRSCs may be produced by an in vitro method comprising: (a) culturing and growing dissociated pRSCs from a mammal in a neural induction medium comprising one or more of an inhibitor of a TGF-3/Activin receptor-like kinases ALK-4, -5 or -7, glycogen synthase kinase-3 (GSK-3), Notch or Wnt signaling or an activator of a hedgehog signaling for a sufficient time to induce pRSCs to a photoreceptor cell lineage fate without visible morphological changes or expression of photoreceptor-specific markers; and (b) followed by, culturing and growing pRSCs of step a) in neural induction medium comprising retinoic acid or taurine or both so as to differentiate the mammalian pRSCs to photoreceptor precursors, thereby producing isolated mammalian photoreceptor precursors; wherein the culture medium is free of feeder cells, feeder-conditioned medium or serum.

The non-neural isolated mammalian retinal pigment epithelial cells (RPECs) may be produced by an in vitro method comprising: (a) culturing pRSCs from a mammal in culture medium comprising nicotinamide or activin A or both in absence of SMAD signaling inhibitor for a sufficient time so as to direct pRSCs toward RPE fate; and (b) culturing the pRSCs in culture medium comprising one or more of a N1 medium supplement, taurine, hydrocortisone, or triiodo-thyronin; so as to differentiate the mammalian pRSCs to mammalian RPECs, thereby, producing isolated mammalian RPECs, wherein the medium is free of feeder cells or feeder-conditioned medium.

Such isolated mammalian OSCs, which can be produced by the described method (see, Zhao, et al, WO 2015/054526) in sufficient quantity and quality, are suitable for cellular transplantation or grafting to an eye of a subject without a need for cellular fractionation or cellular purification prior to cellular transplantation or grafting.

These cells have characteristics of eye field stem cells and can be directed to differentiate toward retinal and corneal lineages in responding to the inductive cues provided in culture. This novel process paves the way to develop off-the-shelf iPSC-derived authentic ocular cell products, which have the advantages of unlimited supply, consistent quality, and no ethical concerns.

Because ocular stem/progenitor and precursor cells are highly expandable and amenable to genetic modifications, they are suitable for rescue and replacing the damaged visual cells and may serve as cellular vehicle to deliver therapeutic drug products to diseased eyes. Ocular fate-restricted cells are of particular interest in this context, as they have been shown to survive for extended periods of time after injection into the vitreous of eyes and to provide retina protection activity on its own. The phase 2b study sponsored by jCyte was designed to evaluate the safety and efficacy of an investigational treatment for RP patients that injected jCells (allogeneic fetal RPCs) intravitreally. Up to 6 million cells were dosed in a single injection. The treatment was well-tolerated, safe, and efficacious in halting, and even reversing, the vision loss among RP patients. Notably, immunosuppression drug was not used in the study (see, Kuppermann, B., “Intravitreal Injection of Allogeneic Human Retinal Progenitor Cells (Jcell) for Treatment of Retinitis Pigmentosa,” The Retina Society. It represents a significant advantage for using progenitor cells over more matured cell products that often require immune suppression.

Two primary considerations in developing an appropriate cell-based therapy are the source of therapeutic cells and the immunological consequences following transplantation. Potential sources of therapeutic differentiated retinal pigmented epithelium (RPE) cells for transplantation studies include pluripotent stem cells (PSCs) derived from embryonic, fetal, or adult cell sources. Recently, the results from early clinical trials of retinal degeneration cell therapy based on four kinds of stem/progenitor cells—RPCs, ESCs, iPSCs, and mesenchymal stem cells (MSCs), suggest that RPCs seem to be the best candidate for the treatment, since it is efficacious and has a shallow risk of rejection and tumorigenesis (Wang Y, et al., Cell Death Dis. 2020 Sep. 23; 11(9):793. doi: 10.1038/s41419-020-02955-3. PMID: 32968042; PMCID: PMC7511341.). However, using fetal donor retina as the source of RPCs has some significant concerns on the limitation of source, scalability and process consistency of manufacture, and ethical conflicts.

The anterior chamber of the eye and the brain have long been described as “immune privileged” sites in transplantation. This phenomenon could have contributed to the tolerance to intraocular transplantation of allogeneic progenitor cells. Besides, cultured murine neural progenitors did not express major histocompatibility complex (MHC) class I or class TT antigens. They were tolerated as allografts even following transplantation to the kidney capsule, a conventional (non-privileged) site.

Although there were some safety concerns about the clinical application of iPSCs due to the potential risk of integration of transgenes into the genome of reprogrammed cells in early years, more recently, small molecules, plasmid vectors, or non-integrating viruses have been utilized for iPSC reprogramming to avoid genomic insertions, thus reducing the risk for translational application and more relevant for clinical applications. The allogeneic iPSC approach could also reduce the cost of iPSC-based cell therapy. It opens a new avenue of retinal disease treatment that could be generally safe, physiologically stable, highly cost-efficient, and targeted at retinal cell revival and regeneration.

The iPSC-derived ocular stem cells can be extensively expanded in culture. These cells, which in analogy to continuously self-renewing embryonic stem cells, can differentiate into retinal neurons and retinal pigment epithelial (RPE) cells in vitro and after transplantation into the retina, and give rise to ocular neural crest stem cells (oNCSCs) and corneal endothelial cells (CECs) in vitro. These cells provide a sustainable source of trophic support for dysfunctional ocular tissues. They may be used as the suitable vehicle and durable reservoir to deliver therapeutic products such as nucleic acids, proteins and other drug moieties to the diseased eye. An advantage of the present inventions' use of the ocular progenitor and precursor cells is that these cells do not need encapsulation to protect themselves from the host immune system.

Sustainable Drug Delivery Approach where Corneal Endothelial Cells are Used for Cell-Mediated Drug Delivery

In some embodiments, corneal endothelial cells (CECs), which can also be derived from ocular stem cells (OSCs) or primitive retinal stem cells (pRSCs) (see, Zhao, et al., WO 2015/054526), may be used as a vehicle to deliver therapeutics in the anterior chamber of the eye for the treatment of corneal diseases. The method of induction of corneal endothelial cells from human pluripotent stem cells have been previously described (see, Zhao, et al, WO 2017/190136A1, the contents of which are incorporated herein in their entirety).

In some embodiments, for the treatment of corneal endothelial diseases, the corneal endothelial cells (CECs) containing a therapeutic agent or drug can be injected into the anterior chamber. This cell therapy approach has been clinically validated for corneal care. In early research and clinical trials in Japan, subjects treated with healthy CECs expanded from a donor cornea have experienced significant and durable improvements in key measures of corneal health including visual acuity, corneal endothelial cell density, and corneal thickness (Kinoshita S, et al., N Engl J Med. 2018 Mar. 15; 378(11):995-1003. Doi: 10.1056/NEJMoa1712770. PMID: 29539291; and Numa K, et al., Ophthalmology. 2021 April; 128(4):504-514. doi: 10.1016/j.ophtha.2020.09.002. Epub 2020 Sep. 6. PMID: 32898516.). One potential application is to treat neovascular cornea, for example, by administration of CECs or iPSC-derived corneal endothelial cells transduced with a viral vector encoding an anti-VEGF product in the anterior chamber.

Corneal Endothelial Cells Derived from Ocular Stem Cells

Corneal endothelial cells (CECs) can be prepared in vitro using the method described in Zhao, et al., WO 2017/190136 A1. Eye cell fate specification was carried out under defined small molecule-driven conditions and in a stepwise fashion of lineage specification. During the initial phase, PSC fate is restricted to the eye field-like state and becomes an ocular stem cell (OSC). In the second phase, PSC-derived OSC is further committed toward retinal lineage or ocular neural crest lineage. The formation of CEnC sheet is directly induced from ocular neural crest stem cell (oNCSC) by suppressing TGF-beta and ROCK signaling in the culture. The induction of oNCSC is initiated by promoting WNT signaling in OSC. Within two weeks of induction, the majority of cells express the typical neural crest markers p75NTR and HNK-1. OSC-derived oNCSCs can be propagated and cryopreserved. Finally, CEC monolayer sheet formation is induced from adherent oNCSCs in the presence of a ROCK inhibitor. The polygonal shaped CEC-like cells become visible after a week in culture. The expression of typical CEC markers such as ZO-1, N-cadherin, and Na+/K+-ATPase is also detected.

Some embodiments of the present inventions include methods of sustainable ocular cell-mediated intraocular delivery of a biological drug into a subject's eye in need of treatment of an ocular disease or disorder with the drug. In certain aspects, such methods comprises a step of (a) preparing a drug expression construct that contains the biological drug coding sequence (e.g., SEQ ID NO: 1, encoding aflibercept) that is linked to a promoter, wherein the biological drug is linked to a leader sequence; (b) introducing the drug expression construct comprising or otherwise based upon a viral vector (e.g., an adeno-associated viral vector or lentiviral vector) or a mammalian plasmid expression vector into ocular cells (e.g., one or more ocular stem cells selected from the group consisting of ocular stem cells, photoreceptor precursors, retinal ganglion precursors, immature RPE cells and/or corneal endothelial cells) in vitro to form engineered ocular cells that can express the biological drug (e.g., aflibercept); (c) formulating engineered ocular cells that are able to produce or express a therapeutic composition; (d) injecting the therapeutic composition comprising the engineered ocular cells into the vitreous, the anterior chamber, the subretinal space, and/or the suprachoroidal space of the subject's eye; (e) causing the production and secretion of the expressed drug (e.g., aflibercept) from the engineered ocular cells in a sustainable manner into the surrounding ocular tissues of the subject, and thereby providing a sustained therapeutic effect for treating the subject's ocular disease or disorder.

Certain embodiments of the present inventions are directed to ocular cells comprising the isolated mammalian ocular stem cells (OSCs) or primitive retinal stem cells (pRSCs), isolated mammalian retinal ganglion precursor cells (RGPCs), isolated mammalian photoreceptor precursor cell (PRPCs), isolated mammalian retinal pigment epithelial cells (RPECs), or isolated mammalian corneal endothelial cells (CECs). These ocular cells can be produced by in vitro methods, for example, as described in Zhao, et al., WO 2015/054526, U.S. Pat. No. 10,220,117 B2, and WO 2017/190136 A1, all of which are incorporated by reference in their entirety herein. For example, in certain embodiments, the ocular cells comprise isolated mammalian pRSCs and can be produced and isolated by: (a) culturing isolated pluripotent stem cells (PSCs) (e.g., embryonic stem cells (ESCs) or induced pluripotent stem cells (iPSCs)) from a mammal in a cell culture medium that is free of feeder cells, feeder-conditioned medium or serum so as to produce and grow a culture of the isolated PSCs; and (b) contacting the culture of the isolated PSCs so grown with one or more of an inhibitor for Wnt or TGF-β/BMP signaling so as to differentiate the isolated PSCs of (a) into primitive retinal stem cells thereby producing isolated mammalian pRSCs, as described in Zhao, et al., WO 2015/054526. In certain embodiments, the ocular cells comprise isolated mammalian RGPCs, which can be produced and isolated by: (a) culturing isolated primitive retinal stem cells (pRSCs) from a mammal in a cell culture medium that is free of feeder cells, feeder-conditioned medium or serum so as to produce and grow a culture of the isolated pRSCs; and (b) contacting the culture of the isolated pRSCs so grown with one or more of an inhibitor of Wnt, Notch, or FGFR/VEGFR signaling so as to differentiate the isolated pRSCs into isolated RGPCs, thereby producing isolated mammalian RGPCs, as also described in Zhao, et al., WO 2015/054526.

In addition, in certain embodiments the mammalian retinal progenitor cells and corneal endothelial cells are directly isolated from donor tissues, for example, fetal retina and cornea donor tissues. These ocular cells can be used as a suitable vehicle and durable reservoir to deliver therapeutic agents or drugs to a diseased eye. Like pluripotent stem cells (PSCs) and other tissue stem cells such as mesenchymal stem cells (MSCs), ocular stem/progenitor and precursor cells normally express low levels of immunogenicity markers. In addition, the eye is a well-known immune privileged organ. Recent data from a Phase 2b clinical trial of intravitreal administration of jCells (human fetal retinal progenitor cells) for RP treatment without immunosuppression drug also confirms the safeness of ocular cells for sustainable drug delivery (see, Kuppermann, B., “Intravitreal Injection of Allogeneic Human Retinal Progenitor Cells (Jcell) for Treatment of Retinitis Pigmentosa,” The Retina Society).

In certain aspects, also disclosed are engineered ocular cell lines and related methods of treatment comprising administering such engineered ocular cell lines to a subject. For example, upon the intravitreal administration of cells from such engineered ocular cell lines to a subject, such cells endogenously express a transgene encoding e.g., a polypeptide (e.g., a polypeptide encoding a VEGF inhibitor) and secrete such polypeptide to thereby cause a sustainable ocular cell-mediated intraocular delivery of such polypeptide (e.g., delivery of such polypeptide into a subject's eye in need of treatment of an ocular disease or disorder with such polypeptide).

As used herein, the term “cell line” refers to a clonal population of cells (e.g., engineer ocular stem cells) that are able to continue to divide and not undergo senescence. In certain embodiments, cells of the engineered ocular cell line endogenously express a transgene encoding a polypeptide (e.g., a polypeptide encoding a VEGF inhibitor), wherein the cells comprise an edited genome that results in the endogenous expression of the transgene, for example, compared to a control cell line. As used herein, the term “control cell line” generally refers to a cell line that is genetically similar to an engineered ocular cell line, but has not been engineered in the same way. For example, an engineered ocular cell line may express an endogenous transgene when compared to a control ocular cell line that is not engineered in the same way. In certain embodiments, the engineered ocular cell line may express an endogenous transgene encoding a VEGF inhibitor (e.g., aflibercept).

In certain embodiments, the ocular cell line comprises ocular stem cells. In certain embodiments, the engineered ocular cell line comprises cell-fate further restricted precursors of ocular stem cells. For example, in certain aspects the ocular cell-fate further restricted precursors of ocular stem cells comprise or are selected from the group consisting of a photoreceptor precursor cell, a retinal ganglion precursor cell, a retinal pigmented epithelial (RPE) cell, a corneal endothelial cell and combinations thereof.

### The Advantage of Ocular Cell-Mediated Delivery Over Other Methods of Delivery of Therapeutic Products

Vascular endothelial growth factor (VEGF) plays an important role in the development of numerous diseases affecting sight, including neovascular age-related macular degeneration (nAMD), diabetic retinopathy, neovascular glaucoma, and neovascular cornea. Intraocular injection of anti-VEGF agents, including pegatanib sodium, ranibizumab (a small anti-VEGF Fab protein which as affinity-improved and made in bacterial E. coli), bevacizumab (a humanized monoclonal antibody against VEGF produced in CHO cells), and aflibercept (a recombinant fusion protein consisting of VEGF-binding regions of the extracellular domains of the human VEGF-receptor fused to the Fc portion of human IgGi, which belongs to a class of molecules commonly known as “VEGF-Traps”) are currently used for treatment of VEGF-associated intraocular conditions.

Since VEGF is chronically produced in a patient's eyes, the neovascular growth comes back when vitreous levels of a VEGF inhibitor drop below a therapeutic level. Thus, current anti-VEGF therapies require repetitive and inconvenient intraocular injections to maintain efficacy, typically ranging from every four to eight weeks in frequency and, in many cases, for the lifetime of the patient. It becomes a substantial treatment burden for patients and their caregivers. Patients often experience vision loss with reduced frequency of treatment due to the compliance lapse. Thus, long-term suppression of pathologic VEGF activity without the requirement for frequent intraocular injections has become a focus of the drug development. For example, a surgical implantation of a trans-scleral device, the PORT DELIVERY SYSTEM (Genentech/Roche), containing a reservoir that is refilled every six months to continuously release a special formulation of Ranibizumab. Although clinical trial results are encouraging, the PORT DELIVERY SYSTEM is implanted in the operating room and requires at least two refills each year, which potentially limits its broad use owing to health care capacity constraints.

Gene therapy has been envisioned as a potential “one-and-done” approach to further decrease the treatment burden. The subject's retinal cells can be transduced with adeno-associated virus (AAV) constructs which can produce the therapeutic molecules continuously inside the eye. Thus, it would eliminate the need for repeated injections of anti-VEGF therapies. Results of early clinical studies, one using a surgical subretinal delivery carried out in the operating room (RGX-314) and the other using an in-office intravitreal injection (ADVM-022), demonstrate the potential of one-time gene therapy to provide long-lasting control of neovascular growth (prnewswire.com/news-releases/regenxbio-presents-positive-interim-data-from-phase-ii-altitude-trial-of-rgx-314-for-the-treatment-of-diabetic-retinopathy-using-suprachoroidal-delivery-301481071.html) (adverum.com/wp-content/uploads/2022/06/Macula-Society-OPTIC-2e11-NAbs-3 Jun. 2022-Final.pdf). However, the ADVM-022 INFINITY study was stopped after a trial participant treated with a high dose of ADVM-022 developed hypotony with inflammation and loss of vision in the treated eye (investors.adverum.com/news/news-details/2021/Adverum-Provides-Update-on-ADVM-022-and-the-INFINITY-Trial-in-Patients-with-Diabetic-Macular-Edema/default.aspx). Several issues could potentially limit the broad use of gene therapy, such as limited and off-targeted delivery and transduction, vulnerability to inflammation, and immune response, coupled with high cost.

The present inventions disclosed herein, including the ocular cell-mediated sustainable intraocular drug delivery approaches described herein, provide safe, effective, stable, and long-term release of therapeutic agents in a subject's eye for treating various eye diseases or disorders. In certain embodiments, the iPSC-derived ocular stem cells (OSCs) are loaded ex vivo with a viral or non-viral drug cargo (a protein or RNA molecules) construct, such as a aflibercept expression cassette as shown in FIG. 1. Once delivered to a subject's eyes, these engineered ocular cells can function as a bio-factory and safe delivery vehicle for the stable and long-term release of the drug in the eye.

The ocular cells are generated by either the direct differentiation from source cells or the isolation and expansion of cells from donor tissues. The oscular stem cells (OSCs) have a low immunogenicity profile. After the intravitreal injection, they can form a self-aggregated and free-floating cell mass. In some embodiments, the cells can be injected into the subretinal space, the transplanted cells can integrate and form a layer of cells in the retina. The cell graft can protect and rescue the adjacent retinal neurons via paracrine signaling and/or regenerate retinal neurons. As described herein, the ocular stem cells are desirable drug carriers. After being loaded with a therapeutic agent or drug (e.g., such as a aflibercept expression cassette as shown in FIG. 1), such ocular stem cells can deliver the drug with retained cell structure and function, such as tissue repair and regeneration with low immunogenicity.

When delivered to a subject's eye(s), the mechanism of action of the engineered ocular cells, which are loaded with a drug in a drug cargo or an expression cassette, comprises (i) expression and/or secretion of the drug cargo in the target area of an eye; (ii) being a stable source of trophic support for dysfunctional ocular tissues; and (iii) being capable of further differentiation to become visual cells of the retina and some of the structural components and connections necessary for sight. Accordingly, in certain embodiments, disclosed herein are methods of treating a subject comprising administering engineered ocular cells to the subject (e.g., administering such engineered ocular cells intraocularly), to thereby cause such cells to express and/or secrete the drug cargo in the target area of an eye of the subject, provide trophic support to the dysfunctional ocular tissues of the subject, cause the further differentiation of such cells to visual cells of the retina and some of the structural components and connections necessary for sight. In certain embodiments, these transplanted ocular cells can rescue and replace the damaged cells. With a low immunogenicity profile, these transplanted ocular cells may provide a long-term release of therapeutic agents, which would eliminate the need for repeated (for example every 6-10 days, or every 4-8 weeks) direct intravitreal rejection of a viral vector carrying a drug as that in other delivery methods, which often have off-target and immunogenicity issues.

As the engineered ocular stem cells are prepared in vitro, and ocular stem cells can be made in large quantity with high efficiency and low cost, they can serve as off-the-shelf ocular cells as an abundant source for using as a cell-mediated drug carrier in the disclosed cell therapy approach. Thus, the present invention and the off-the-shelf ocular cell therapy approaches disclosed herein have a significant advantage over the current gene therapy approaches, in that ocular cell therapy could target a large population of patients regardless of the underlying disease-causing genetic mutations, while the current gene therapy may only target the patients carrying a specific mutated gene. These patients usually represent a small portion of the inherited retinal degenerative disease patient population, for example, patients with RPE65 gene mutations only count for about 2% of Retinitis Pigmentosa patients. In addition, the engineered cells can be extensively characterized in vitro for its safety and efficacy prior to clinical development.

The presently described sustained ocular cell-mediated intraocular delivery of cellular therapeutics and the treatment approaches disclosed herein can not only protect and rescue retinal tissue via paracrine signaling and molecular exchanges between the grafted healthy cells and dying host retinal cells, but also are able to integrate and regenerate new RPE and photoreceptor cells. The foregoing represents a significant advantage over the RPE cell transplantation methods currently in development for treating geographic atrophy AMD due to progressive degeneration of RPE cells and photoreceptors. Because the RPE cell transplantation method mainly provides trophic protection for the remaining photoreceptors, no cell regeneration involved, and not suitable for the advanced stage patients, who only has a few residual photoreceptors left in the eye.

In some embodiments, the ocular stem cells transduced with lentiviral or AAV cargo constructs can ensure high and long-last expression of a vascular endothelial growth factor inhibitor for treating Wet AMD and Diabetic Retinopathy. The dose can be pre-determined and can be delivered safely without a surgical procedure for global delivery without local gradient effect. This sustainable ocular cell-mediated intraocular delivery of cellular therapeutic approach has apparent advantages over the direct injection methods for delivering the Anti-VEGF Abs or VEGF-trap, or potentially other biological injectable drugs which requires frequent doses every 4-6 weeks on average. This sustainable approach of the ocular cell-mediated intraocular delivery of a therapeutic agent or drug has apparent advantage over the AAV-based gene therapy, which is serotype with dose limited infection efficiency, and has procedure related adverse events, and immunogenic responses.

### Therapeutic Agents or Drugs and Compositions

The therapeutic agent, biologic or drug, which can be delivered to a diseased eye to treat various eye diseases or disorders in accordance with the present inventions and ocular stem cell-mediated intraocular delivery methods disclosed herein, include any therapeutic agents, biologics, drugs or products, such as, for example, nucleic acids, proteins, and other drug rnoieties, that can treat eye diseases or disorders. In certain embodiments, such therapeutic agent comprises aflibercept, as exemplified in FIG. 1. Aflibercept, sold under the brand names EYLEA and ZALTRAP, is a drug used to treat wet macular degeneration and metastatic colorectal cancer. The amino acid sequence (no signal peptide) of aflibercept (SEQ ID NO: 1), and the Codon optimized nucleic acid sequence encoding aflibercept (SEQ ID NO: 2) (IL2 leader sequence is underlined) are shown in Table 3 below.

TABLE 3

| ID | Sequence | SEQ ID NO: |
|---|---|---|
| Aflibercept | SDTGRPFVEMYSEIPEIIHMTEGRELVIPCRVTSPNITVTLKKFPLDTLIPDG | 1 |
| amino acid | KRIIWDSRKGFIISNATYKEIGLLTCEATVNGHLYKTNYLTHRQTNTIIDVVL | |
| sequence (no | SPSHGIELSVGEKLVLNCTARTELNVGIDENWEYPSSKHQHKKLVNRDLKTQS | |
| signal | GSEMKKFLSTLTIDGVTRSDQGLYTCAASSGLMTKKNSTFVRVHEKDKTHTCP | |
| peptide) | PCPAPELLGGPSVFLFPPKPKDTLMISRTPEVTCVVVDVSHEDPEVKENWYVD | |
| | GVEVHNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWESNGQ | |
| | PENNYKTTPPVLDSDGSFFLYSKLTVDKSRWQQGNVFSCSVMHEALHNHYTQK | |
| | SLSLSP | |
| Codon | AGATCATCCACATGACCGAGGGCAGAGAGCTGGTGATCCCCTGCAGAGTGACC | 2 |
| optimized | GAATTCGAGCGACACCGGCAGACCCTTCGTGGAGATGTACAGCGAGATCCCCG | |
| nucleic acid | AGATCATCCACATGACCGAGGGCAGAGAGCTGGTGATCCCCTGCAGAGTGACC | |
| sequence | AGCCCCAACATCACCGTGACCCTGAAGAAGTTCCCCCTGGACACCCTGATCCC | |
| encoding | CGACGGCAAGAGAATCATCTGGGACAGCAGAAAGGGCTTCATCATCAGCAACG | |
| aflibercept | CCACCTACAAGGAGATCGGCCTGCTGACCTGCGAGGCCACCGTGAACGGCCAC | |
| (IL2 leader | CTGTACAAGACCAACTACCTGACCCACAGACAGACCAACACCATCATCGACGT | |
| sequence is | GGTGCTGAGCCCCAGCCACGGCATCGAGCTGAGCGTGGGCGAGAAGCTGGTGC | |
| underlined) | TGAACTGCACCGCCAGAACCGAGCTGAACGTGGGCATCGACTTCAACTGGGAG | |
| | TACCCCAGCAGCAAGCACCAGCACAAGAAGCTGGTGAACAGAGACCTGAAGAC | |
| | CCAGAGCGGCAGCGAGATGAAGAAGTTCCTGAGCACCCTGACCATCGACGGCG | |
| | TGACCAGAAGCGACCAGGGCCTGTACACCTGCGCCGCCAGCAGCGGCCTGATG | |
| | ACCAAGAAGAACAGCACCTTCGTGAGAGTGCACGAGAAGGACAAGACCCACAC | |
| | CTGCCCCCCCTGCCCCGCCCCCGAGCTGCTGGGCGGCCCCAGCGTGTTCCTGT | |
| | TCCCCCCCAAGCCCAAGGACACCCTGATGATCAGCAGAACCCCCGAGGTGACC | |
| | TGCGTGGTGGTGGACGTGAGCCACGAGGACCCCGAGGTGAAGTTCAACTGGTA | |
| | CGTGGACGGCGTGGAGGTGCACAACGCCAAGACCAAGCCCAGAGAGGAGCAGT | |
| | ACAACAGCACCTACAGAGTGGTGAGCGTGCTGACCGTGCTGCACCAGGACTGG | |
| | CTGAACGGCAAGGAGTACAAGTGCAAGGTGAGCAACAAGGCCCTGCCCGCCCC | |
| | CATCGAGAAGACCATCAGCAAGGCCAAGGGCCAGCCCAGAGAGCCCCAGGTGT | |
| | ACACCCTGCCCCCCAGCAGAGACGAGCTGACCAAGAACCAGGTGAGCCTGACC | |
| | TGCCTGGTGAAGGGCTTCTACCCCAGCGACATCGCCGTGGAGTGGGAGAGCAA | |
| | CGGCCAGCCCGAGAACAACTACAAGACCACCCCCCCCGTGCTGGACAGCGACG | |
| | GCAGCTTCTTCCTGTACAGCAAGCTGACCGTGGACAAGAGCAGATGGCAGCAG | |
| | GGCAACGTGTTCAGCTGCAGCGTGATGCACGAGGCCCTGCACAACCACTACAC | |
| | CCAGAAGAGCCTGAGCCTGAGCCCC | |

Ocular-fate restricted cells can be engineered in vitro to enable them to produce drugs with therapeutic effects, such as aflibercept, to treat neovascular ocular conditions. These cells can then be injected into the vitreous, the anterior chamber, the suprachoroidal space, or the subretinal space as shown in FIG. 1. They continuously produce and release drug molecules to the surrounding ocular tissues. A therapeutic amount of a drug, such as aflibercept, may be administered via ocular stem cell-mediated intraocular delivery.

As used herein, the phrase “therapeutically effective amount” means an amount sufficient to prevent, treat, reduce the severity of, delay the onset of, or inhibit a symptom of a disease. The symptom can be a symptom already occurring or expected to occur. Determination of a therapeutically effective amount is well within the capability of those skilled in the art, especially in light of the detailed disclosure provided herein.

The therapeutic agent or drug, such as aflibercept, which may be carried by ocular cells for cell-mediated drug delivery, as described herein, may be administered at a suitable dose, e.g., at a suitable volume and concentration, which may be about 100,000 cells to about 10,000,000 cells in a volume of 10 μL to 300 μL, about 100,000 cells to about 500,000 cells in a volume of 10 μL to 300 μL, about 500,000 cells to about 600,000 cells in a volume of 10 μL to 300 μL, about 600,000 cells to about 700,000 cells in a volume of 10 μL to 300 μL, about 700,000 cells to about 800,000 cells in a volume of 10 μL to 300 μL, about 800,000 cells to about 900,000 cells in a volume of 10 μL to 300 μL, about 900,000 cells to about 1,000,000 cells in a volume of 10 μL to 300 μL, about 1,000,000 cells to about 2,000,000 cells in a volume of 10 μL to 300 μL, about 2,000,000 cells to about 3,000,000 cells in a volume of 10 μL to 300 μL, about 3,000,000 cells to about 4,000,000 cells in a volume of 10 μL to 300 μL, about 4,000,000 cells to about 5,000,000 cells in a volume of 10 μL to 300 μL, about 500,000 cells in a volume of 10 μL to 300 μL, about 5,000,000 cells in a volume of 10 μL to 300 μL, about 5,000,000 cells to about 6,000,000 cells in a volume of 10 μL to 300 μL, about 6,000,000 cells to about 10,000,000 cells in a volume of 10 μL to 300 μL, at least about 100,000 cells in a volume of 10 μL to 300 μL, or up to 10,000,000 cells in a volume of 10 μL to 300 μL, or any volume and concentration in a range bounded by, or between, any of these values.

### Pharmaceutical Compositions

According to the present disclosure the compositions (e.g., ocular stem cells expressing a therapeutic agent or drug, such as aflibercept) may be prepared as pharmaceutical compositions (e.g., a pharmaceutical composition for ophthalmic or intraocular administration). It will be understood that such compositions necessarily comprise one or more active ingredients and, most often, a pharmaceutically acceptable excipient. Relative amounts of the active ingredient (e.g., ocular stem cells expressing a therapeutic agent or drug, such as aflibercept), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

Although the descriptions of pharmaceutical compositions provided herein are principally directed to pharmaceutical compositions which are suitable for administration to humans, it will be understood by the skilled artisan that such compositions are generally suitable for administration to any other animal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Patients and subjects to which administration of the pharmaceutical compositions is contemplated include, but are not limited to, humans and/or other primates. In some embodiments, compositions are administered to humans, human patients or subjects.

The compositions disclosed herein can be formulated using one or more excipients. Formulations of the present disclosure can include, without limitation, saline, liposomes, lipid nanoparticles, polymers, peptides, proteins, cells and combinations thereof. Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients. In general, such preparatory methods include the step of associating the active ingredient with an excipient and/or one or more other accessory ingredients.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see, Remington: The Science and Practice of Pharmacy, 21st Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In certain embodiments, the compositions of the present disclosure may be delivered by intraocular delivery route. A non-limiting example of intraocular administration include an intravitreal injection. Accordingly, in some embodiments, the pharmaceutical compositions of the present disclosure may be prepared, packaged, and/or sold in formulations suitable for ophthalmic administration. Such formulations may, for example, be in the form of eye drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

### Ocular Diseases

In some embodiments, the compositions and methods of the present disclosure may be used to treat subjects having or suspected of developing an ocular disease. The eye is an organ comprising a number of components, including the cornea, aqueous humor, lens, vitreous humor, retina, the retinal pigment epithelium, and choroid. Ocular diseases are conditions affecting the different tissues of the eye. The inventions disclosed herein are suitable for the treatment of a number of diseases and disorders affecting the eye or the different components of the eye, including, for example impaired vision, full or partial blindness, irritation, dryness, sensitivity, photophobia, and/or light aversion.

In certain embodiments, the inventions disclosed herein are suitable for the treatment of age-related macular degeneration (AMD). AMD is a major cause of irreversible loss of central vision in the elderly worldwide. AMD leads to gradually worsening vision. AMD does not result in blindness, but may affect daily life. Wet AMD is caused by abnormal blood vessels behind the retina grow under the macula and leak blood and fluid that damage the macula. Wet AMD may be treated with laser coagulation and medication to reverse or stop the growth of blood vessels. Dry AMD is caused by break down of the light sensitive cells in the macula. There are currently no treatments for dry AMD. There remains a need for prevention, management and treatment therapies for wet and dry AMD. AMD is associated with complement components. In addition, AMD is associated with proteins such as, but not limited to, VEGF (Vascular endothelial growth factor), EPO (Erythropoietin), EPOR (EPO receptor), Interleukins IL-1β, IL-17A, 11-10, TNFα (tumor necrosis factor alpha), or FGFR2 (Fibroblast Growth Factor Receptor).

In certain embodiments, the inventions disclosed herein are suitable for the treatment of corneal diseases. Corneal diseases affect the cornea and the conjunctiva. Cornea and conjunctiva form the outer surface of the eye, which is exposed to external environment, and are susceptible to infection agents, trauma, and/or exposure to chemicals, toxins, allergens, etc. The cornea is also affected by autoimmune conditions, nutritional deficiencies and cancer. Corneal diseases may cause for example, loss of vision, blurred vision, tearing, light sensitivity and pain. Diseases affecting cornea include, but are not limited to, keratitis, corneal dystrophy, corneal degeneration, Fuchs' dystrophy, cancer of cornea, and keratoconjuctivitis. Though surgical and medical treatment therapies for corneal diseases exist, in some cases, the diseases still remain severe and may cause blindness.

In certain embodiments, the inventions disclosed herein are suitable for the treatment of Uveitis. Uveitis is an inflammation of the uvea, comprising the iris, choroids, and ciliary body. Early symptoms include eye redness, pain, irritation and blurred vision. Uveitis may lead to transient or permanent loss of vision. Uveitis may be associated with other diseases and conditions, such as infections, systemic diseases, non-infectious and autoimmune diseases.

In certain embodiments, the inventions disclosed herein are suitable for the treatment of retinopathy. Retinopathy is a disease resulting from neovascularization (excessive growth of blood vessels) in the light-sensitive tissue of the eye, retina. Retinopathy may result in impaired vision or partial or full blindness. Retinopathy may be caused by systemic diseases, e.g., diabetes, or hypertension, trauma, excessive sun light exposure or ionizing radiation. Retinopathy is often treated with laser therapy. There remains a need for new therapies for prevention, management and/or treatment of retinopathy.

The present disclosure is further illustrated by the following non-limiting examples.

### EXAMPLES

### Example 1

### Construction of Aflibercept Expression Vector ABDNA202:

The expression of the aflibercept coding sequence was driven by the human eukaryotic translation elongation factor 1 alpha1 short form promoter (EFS) in the mammalian gene expression lentiviral vector identified below in Table 4 as SEQ ID NO: 10. The vector also carries a mCherry marker expression cassette under the control of the CMV promoter.

TABLE 4

| ID | Sequence | SEQ ID NO: |
|---|---|---|
| Aflibercept | AATGTAGTCTTATGCAATACTCTTGTAGTCTTGCAACATGGTAACGATGAGTT | 10 |
| lentiviral | AGCAACATGCCTTACAAGGAGAGAAAAAGCACCGTGCATGCCGATTGGTGGAA | |
| expression | GTAAGGTGGTACGATCGTGCCTTATTAGGAAGGCAACAGACGGGTCTGACATG | |
| construct | GATTGGACGAACCACTGAATTGCCGCATTGCAGAGATATTGTATTTAAGTGCC | |
| | TAGCTCGATACATAAACGGGTCTCTCTGGTTAGACCAGATCTGAGCCTGGGAG | |
| | CTCTCTGGCTAACTAGGGAACCCACTGCTTAAGCCTCAATAAAGCTTGCCTTG | |
| | AGTGCTTCAAGTAGTGTGTGCCCGTCTGTTGTGTGACTCTGGTAACTAGAGAT | |
| | CCCTCAGACCCTTTTAGTCAGTGTGGAAAATCTCTAGCAGTGGCGCCCGAACA | |
| | GGGACTTGAAAGCGAAAGGGAAACCAGAGGAGCTCTCTCGACGCAGGACTCGG | |
| | CTTGCTGAAGCGCGCACGGCAAGAGGCGAGGGGCGGCGACTGGTGAGTACGCC | |
| | AAAAATTTTGACTAGCGGAGGCTAGAAGGAGAGAGATGGGTGCGAGAGCGTCA | |
| | GTATTAAGCGGGGGAGAATTAGATCGCGATGGGAAAAAATTCGGTTAAGGCCA | |
| | GGGGGAAAGAAAAAATATAAATTAAAACATATAGTATGGGCAAGCAGGGAGCT | |
| | AGAACGATTCGCAGTTAATCCTGGCCTGTTAGAAACATCAGAAGGCTGTAGAC | |
| | AAATACTGGGACAGCTACAACCATCCCTTCAGACAGGATCAGAAGAACTTAGA | |
| | TCATTATATAATACAGTAGCAACCCTCTATTGTGTGCATCAAAGGATAGAGAT | |
| | AAAAGACACCAAGGAAGCTTTAGACAAGATAGAGGAAGAGCAAAACAAAAGTA | |
| | AGACCACCGCACAGCAAGCGGCCGCTGATCTTCAGACCTGGAGGAGGAGATAT | |
| | GAGGGACAATTGGAGAAGTGAATTATATAAATATAAAGTAGTAAAAATTGAAC | |
| | CATTAGGAGTAGCACCCACCAAGGCAAAGAGAAGAGTGGTGCAGAGAGAAAAA | |
| | AGAGCAGTGGGAATAGGAGCTTTGTTCCTTGGGTTCTTGGGAGCAGCAGGAAG | |
| | CACTATGGGCGCAGCGTCAATGACGCTGACGGTACAGGCCAGACAATTATTGT | |
| | CTGGTATAGTGCAGCAGCAGAACAATTTGCTGAGGGCTATTGAGGCGCAACAG | |
| | CATCTGTTGCAACTCACAGTCTGGGGCATCAAGCAGCTCCAGGCAAGAATCCT | |
| | GGCTGTGGAAAGATACCTAAAGGATCAACAGCTCCTGGGGATTTGGGGTTGCT | |
| | CTGGAAAACTCATTTGCACCACTGCTGTGCCTTGGAATGCTAGTTGGAGTAAT | |
| | AAATCTCTGGAACAGATTTGGAATCACACGACCTGGATGGAGTGGGACAGAGA | |
| | AATTAACAATTACACAAGCTTAATACACTCCTTAATTGAAGAATCGCAAAACC | |
| | AGCAAGAAAAGAATGAACAAGAATTATTGGAATTAGATAAATGGGCAAGTTTG | |
| | TGGAATTGGTTTAACATAACAAATTGGCTGTGGTATATAAAATTATTCATAAT | |
| | GATAGTAGGAGGCTTGGTAGGTTTAAGAATAGTTTTTGCTGTACTTTCTATAG | |
| | TGAATAGAGTTAGGCAGGGATATTCACCATTATCGTTTCAGACCCACCTCCCA | |
| | ACCCCGAGGGGACCCGACAGGCCCGAAGGAATAGAAGAAGAAGGTGGAGAGAG | |
| | AGACAGAGACAGATCCATTCGATTAGTGAACGGATCTCGACGGTATCGCTAGC | |
| | TTTTAAAAGAAAAGGGGGGATTGGGGGGTACAGTGCAGGGGAAAGAATAGTAG | |
| | ACATAATAGCAACAGACATACAAACTAAAGAATTACAAAAACAAATTACAAAA | |
| | ATTCAAAATTTTACTAGTGATTATCGGATCAACTTTGTATAGAAAAGTTGGGC | |
| | TCCGGTGCCCGTCAGTGGGCAGAGCGCACATCGCCCACAGTCCCCGAGAAGTT | |
| | GGGGGGAGGGGTCGGCAATTGATCCGGTGCCTAGAGAAGGTGGCGCGGGGTAA | |
| | ACTGGGAAAGTGATGTCGTGTACTGGCTCCGCCTTTTTCCCGAGGGTGGGGGA | |
| | GAACCGTATATAAGTGCAGTAGTCGCCGTGAACGTTCTTTTTCGCAACGGGTT | |
| | TGCCGCCAGAACACAGGCAAGTTTGTACAAAAAAGCAGGCTGCCACCATGTAC | |
| | AGGATGCAACTCCTGTCTTGCATTGCACTAAGTCTTGCACTTGTCACGAATTC | |
| | GAGCGACACCGGCAGACCCTTCGTGGAGATGTACAGCGAGATCCCCGAGATCA | |
| | TCCACATGACCGAGGGCAGAGAGCTGGTGATCCCCTGCAGAGTGACCAGCCCC | |
| | AACATCACCGTGACCCTGAAGAAGTTCCCCCTGGACACCCTGATCCCCGACGG | |
| | CAAGAGAATCATCTGGGACAGCAGAAAGGGCTTCATCATCAGCAACGCCACCT | |
| | ACAAGGAGATCGGCCTGCTGACCTGCGAGGCCACCGTGAACGGCCACCTGTAC | |
| | AAGACCAACTACCTGACCCACAGACAGACCAACACCATCATCGACGTGGTGCT | |
| | GAGCCCCAGCCACGGCATCGAGCTGAGCGTGGGCGAGAAGCTGGTGCTGAACT | |
| | GCACCGCCAGAACCGAGCTGAACGTGGGCATCGACTTCAACTGGGAGTACCCC | |
| | AGCAGCAAGCACCAGCACAAGAAGCTGGTGAACAGAGACCTGAAGACCCAGAG | |
| | CGGCAGCGAGATGAAGAAGTTCCTGAGCACCCTGACCATCGACGGCGTGACCA | |
| | GAAGCGACCAGGGCCTGTACACCTGCGCCGCCAGCAGCGGCCTGATGACCAAG | |
| | AAGAACAGCACCTTCGTGAGAGTGCACGAGAAGGACAAGACCCACACCTGCCC | |
| | CCCCTGCCCCGCCCCCGAGCTGCTGGGCGGCCCCAGCGTGTTCCTGTTCCCCC | |
| | CCAAGCCCAAGGACACCCTGATGATCAGCAGAACCCCCGAGGTGACCTGCGTG | |
| | GTGGTGGACGTGAGCCACGAGGACCCCGAGGTGAAGTTCAACTGGTACGTGGA | |
| | CGGCGTGGAGGTGCACAACGCCAAGACCAAGCCCAGAGAGGAGCAGTACAACA | |
| | GCACCTACAGAGTGGTGAGCGTGCTGACCGTGCTGCACCAGGACTGGCTGAAC | |
| | GGCAAGGAGTACAAGTGCAAGGTGAGCAACAAGGCCCTGCCCGCCCCCATCGA | |
| | GAAGACCATCAGCAAGGCCAAGGGCCAGCCCAGAGAGCCCCAGGTGTACACCC | |
| | TGCCCCCCAGCAGAGACGAGCTGACCAAGAACCAGGTGAGCCTGACCTGCCTG | |
| | GTGAAGGGCTTCTACCCCAGCGACATCGCCGTGGAGTGGGAGAGCAACGGCCA | |
| | GCCCGAGAACAACTACAAGACCACCCCCCCCGTGCTGGACAGCGACGGCAGCT | |
| | TCTTCCTGTACAGCAAGCTGACCGTGGACAAGAGCAGATGGCAGCAGGGCAAC | |
| | GTGTTCAGCTGCAGCGTGATGCACGAGGCCCTGCACAACCACTACACCCAGAA | |
| | GAGCCTGAGCCTGAGCCCCTGAACCCAGCTTTCTTGTACAAAGTGGTGATAAT | |
| | CGAATTCCGATAATCAACCTCTGGATTACAAAATTTGTGAAAGATTGACTGGT | |
| | ATTCTTAACTATGTTGCTCCTTTTACGCTATGTGGATACGCTGCTTTAATGCC | |
| | TTTGTATCATGCTATTGCTTCCCGTATGGCTTTCATTTTCTCCTCCTTGTATA | |
| | AATCCTGGTTGCTGTCTCTTTATGAGGAGTTGTGGCCCGTTGTCAGGCAACGT | |
| | GGCGTGGTGTGCACTGTGTTTGCTGACGCAACCCCCACTGGTTGGGGCATTGC | |
| | CACCACCTGTCAGCTCCTTTCCGGGACTTTCGCTTTCCCCCTCCCTATTGCCA | |
| | CGGCGGAACTCATCGCCGCCTGCCTTGCCCGCTGCTGGACAGGGGCTCGGCTG | |
| | TTGGGCACTGACAATTCCGTGGTGTTGTCGGGGAAGCTGACGTCCTTTCCATG | |
| | GCTGCTCGCCTGTGTTGCCACCTGGATTCTGCGCGGGACGTCCTTCTGCTACG | |
| | TCCCTTCGGCCCTCAATCCAGCGGACCTTCCTTCCCGCGGCCTGCTGCCGGCT | |
| | CTGCGGCCTCTTCCGCGTCTTCGCCTTCGCCCTCAGACGAGTCGGATCTCCCT | |
| | TTGGGCCGCCTCCCCGCATCGGGAATTCCCGCGGTTCGAACGCGTTGACATTG | |
| | ATTATTGACTAGTTATTAATAGTAATCAATTACGGGGTCATTAGTTCATAGCC | |
| | CATATATGGAGTTCCGCGTTACATAACTTACGGTAAATGGCCCGCCTGGCTGA | |
| | CCGCCCAACGACCCCCGCCCATTGACGTCAATAATGACGTATGTTCCCATAGT | |
| | AACGCCAATAGGGACTTTCCATTGACGTCAATGGGTGGAGTATTTACGGTAAA | |
| | CTGCCCACTTGGCAGTACATCAAGTGTATCATATGCCAAGTACGCCCCCTATT | |
| | GACGTCAATGACGGTAAATGGCCCGCCTGGCATTATGCCCAGTACATGACCTT | |
| | ATGGGACTTTCCTACTTGGCAGTACATCTACGTATTAGTCATCGCTATTACCA | |
| | TGGTGATGCGGTTTTGGCAGTACATCAATGGGCGTGGATAGCGGTTTGACTCA | |
| | CGGGGATTTCCAAGTCTCCACCCCATTGACGTCAATGGGAGTTTGTTTTGGCA | |
| | CCAAAATCAACGGGACTTTCCAAAATGTCGTAACAACTCCGCCCCATTGACGC | |
| | AAATGGGCGGTAGGCGTGTACGGTGGGAGGTCTATATAAGCAGAGCTCTCTGG | |
| | CTAACTAGAGAACCCACTGCGCCACCATGGTGAGCAAGGGCGAGGAGGATAAC | |
| | ATGGCCATCATCAAGGAGTTCATGCGCTTCAAGGTGCACATGGAGGGCTCCGT | |
| | GAACGGCCACGAGTTCGAGATCGAGGGCGAGGGCGAGGGCCGCCCCTACGAGG | |
| | GCACCCAGACCGCCAAGCTGAAGGTGACCAAGGGTGGCCCCCTGCCCTTCGCC | |
| | TGGGACATCCTGTCCCCTCAGTTCATGTACGGCTCCAAGGCCTACGTGAAGCA | |
| | CCCCGCCGACATCCCCGACTACTTGAAGCTGTCCTTCCCCGAGGGCTTCAAGT | |
| | GGGAGCGCGTGATGAACTTCGAGGACGGCGGCGTGGTGACCGTGACCCAGGAC | |
| | TCCTCCCTGCAGGACGGCGAGTTCATCTACAAGGTGAAGCTGCGCGGCACCAA | |
| | CTTCCCCTCCGACGGCCCCGTAATGCAGAAGAAGACCATGGGCTGGGAGGCCT | |
| | CCTCCGAGCGGATGTACCCCGAGGACGGCGCCCTGAAGGGCGAGATCAAGCAG | |
| | AGGCTGAAGCTGAAGGACGGCGGCCACTACGACGCTGAGGTCAAGACCACCTA | |
| | CAAGGCCAAGAAGCCCGTGCAGCTGCCCGGCGCCTACAACGTCAACATCAAGT | |
| | TGGACATCACCTCCCACAACGAGGACTACACCATCGTGGAACAGTACGAACGC | |
| | GCCGAGGGCCGCCACTCCACCGGCGGCATGGACGAGCTGTACAAGTAAGGTAC | |
| | CTTTAAGACCAATGACTTACAAGGCAGCTGTAGATCTTAGCCACTTTTTAAAA | |
| | GAAAAGGGGGGACTGGAAGGGCTAATTCACTCCCAACGAAGACAAGATCTGCT | |
| | TTTTGCTTGTACTGGGTCTCTCTGGTTAGACCAGATCTGAGCCTGGGAGCTCT | |
| | CTGGCTAACTAGGGAACCCACTGCTTAAGCCTCAATAAAGCTTGCCTTGAGTG | |
| | CTTCAAGTAGTGTGTGCCCGTCTGTTGTGTGACTCTGGTAACTAGAGATCCCT | |
| | CAGACCCTTTTAGTCAGTGTGGAAAATCTCTAGCAGTAGTAGTTCATGTCATC | |
| | TTATTATTCAGTATTTATAACTTGCAAAGAAATGAATATCAGAGAGTGAGAGG | |
| | AACTTGTTTATTGCAGCTTATAATGGTTACAAATAAAGCAATAGCATCACAAA | |
| | TTTCACAAATAAAGCATTTTTTTCACTGCATTCTAGTTGTGGTTTGTCCAAAC | |
| | TCATCAATGTATCTTATCATGTCTGGCTCTAGCTATCCCGCCCCTAACTCCGC | |
| | CCATCCCGCCCCTAACTCCGCCCAGTTCCGCCCATTCTCCGCCCCATGGCTGA | |
| | CTAATTTTTTTTATTTATGCAGAGGCCGAGGCCGCCTCGGCCTCTGAGCTATT | |
| | CCAGAAGTAGTGAGGAGGCTTTTTTGGAGGCCTAGGGACGTACCCAATTCGCC | |
| | CTATAGTGAGTCGTATTACGCGCGCTCACTGGCCGTCGTTTTACAACGTCGTG | |
| | ACTGGGAAAACCCTGGCGTTACCCAACTTAATCGCCTTGCAGCACATCCCCCT | |
| | TTCGCCAGCTGGCGTAATAGCGAAGAGGCCCGCACCGATCGCCCTTCCCAACA | |
| | GTTGCGCAGCCTGAATGGCGAATGGGACGCGCCCTGTAGCGGCGCATTAAGCG | |
| | CGGCGGGTGTGGTGGTTACGCGCAGCGTGACCGCTACACTTGCCAGCGCCCTA | |
| | GCGCCCGCTCCTTTCGCTTTCTTCCCTTCCTTTCTCGCCACGTTCGCCGGCTT | |
| | TCCCCGTCAAGCTCTAAATCGGGGGCTCCCTTTAGGGTTCCGATTTAGTGCTT | |
| | TACGGCACCTCGACCCCAAAAAACTTGATTAGGGTGATGGTTCACGTAGTGGG | |
| | CCATCGCCCTGATAGACGGTTTTTCGCCCTTTGACGTTGGAGTCCACGTTCTT | |
| | TAATAGTGGACTCTTGTTCCAAACTGGAACAACACTCAACCCTATCTCGGTCT | |
| | ATTCTTTTGATTTATAAGGGATTTTGCCGATTTCGGCCTATTGGTTAAAAAAT | |
| | GAGCTGATTTAACAAAAATTTAACGCGAATTTTAACAAAATATTAACGCTTAC | |
| | AATTTAGGTGGCACTTTTCGGGGAAATGTGCGCGGAACCCCTATTTGTTTATT | |
| | TTTCTAAATACATTCAAATATGTATCCGCTCATGAGACAATAACCCTGATAAA | |
| | TGCTTCAATAATATTGAAAAAGGAAGAGTATGAGTATTCAACATTTCCGTGTC | |
| | GCCCTTATTCCCTTTTTTGCGGCATTTTGCCTTCCTGTTTTTGCTCACCCAGA | |
| | AACGCTGGTGAAAGTAAAAGATGCTGAAGATCAGTTGGGTGCACGAGTGGGTT | |
| | ACATCGAACTGGATCTCAACAGCGGTAAGATCCTTGAGAGTTTTCGCCCCGAA | |
| | GAACGTTTTCCAATGATGAGCACTTTTAAAGTTCTGCTATGTGGCGCGGTATT | |
| | ATCCCGTATTGACGCCGGGCAAGAGCAACTCGGTCGCCGCATACACTATTCTC | |
| | AGAATGACTTGGTTGAGTACTCACCAGTCACAGAAAAGCATCTTACGGATGGC | |
| | ATGACAGTAAGAGAATTATGCAGTGCTGCCATAACCATGAGTGATAACACTGC | |
| | GGCCAACTTACTTCTGACAACGATCGGAGGACCGAAGGAGCTAACCGCTTTTT | |
| | TGCACAACATGGGGGATCATGTAACTCGCCTTGATCGTTGGGAACCGGAGCTG | |
| | AATGAAGCCATACCAAACGACGAGCGTGACACCACGATGCCTGTAGCAATGGC | |
| | AACAACGTTGCGCAAACTATTAACTGGCGAACTACTTACTCTAGCTTCCCGGC | |
| | AACAATTAATAGACTGGATGGAGGCGGATAAAGTTGCAGGACCACTTCTGCGC | |
| | TCGGCCCTTCCGGCTGGCTGGTTTATTGCTGATAAATCTGGAGCCGGTGAGCG | |
| | TGGGTCTCGCGGTATCATTGCAGCACTGGGGCCAGATGGTAAGCCCTCCCGTA | |
| | TCGTAGTTATCTACACGACGGGGAGTCAGGCAACTATGGATGAACGAAATAGA | |
| | CAGATCGCTGAGATAGGTGCCTCACTGATTAAGCATTGGTAACTGTCAGACCA | |
| | AGTTTACTCATATATACTTTAGATTGATTTAAAACTTCATTTTTAATTTAAAA | |
| | GGATCTAGGTGAAGATCCTTTTTGATAATCTCATGACCAAAATCCCTTAACGT | |
| | GAGTTTTCGTTCCACTGAGCGTCAGACCCCGTAGAAAAGATCAAAGGATCTTC | |
| | TTGAGATCCTTTTTTTCTGCGCGTAATCTGCTGCTTGCAAACAAAAAAACCAC | |
| | CGCTACCAGCGGTGGTTTGTTTGCCGGATCAAGAGCTACCAACTCTTTTTCCG | |
| | AAGGTAACTGGCTTCAGCAGAGCGCAGATACCAAATACTGTTCTTCTAGTGTA | |
| | GCCGTAGTTAGGCCACCACTTCAAGAACTCTGTAGCACCGCCTACATACCTCG | |
| | CTCTGCTAATCCTGTTACCAGTGGCTGCTGCCAGTGGCGATAAGTCGTGTCTT | |
| | ACCGGGTTGGACTCAAGACGATAGTTACCGGATAAGGCGCAGCGGTCGGGCTG | |
| | AACGGGGGGTTCGTGCACACAGCCCAGCTTGGAGCGAACGACCTACACCGAAC | |
| | TGAGATACCTACAGCGTGAGCTATGAGAAAGCGCCACGCTTCCCGAAGAGAGA | |
| | AAGGCGGACAGGTATCCGGTAAGCGGCAGGGTCGGAACAGGAGAGCGCACGAG | |
| | GGAGCTTCCAGGGGGAAACGCCTGGTATCTTTATAGTCCTGTCGGGTTTCGCC | |
| | ACCTCTGACTTGAGCGTCGATTTTTGTGATGCTCGTCAGGGGGGCGGAGCCTA | |
| | TGGAAAAACGCCAGCAACGCGGCCTTTTTACGGTTCCTGGCCTTTTGCTGGCC | |
| | TTTTGCTCACATGTTCTTTCCTGCGTTATCCCCTGATTCTGTGGATAACCGTA | |
| | TTACCGCCTTTGAGTGAGCTGATACCGCTCGCCGCAGCCGAACGACCGAGCGC | |
| | AGCGAGTCAGTGAGCGAGGAAGCGGAAGAGCGCCCAATACGCAAACCGCCTCT | |
| | CCCCGCGCGTTGGCCGATTCATTAATGCAGCTGGCACGACAGGTTTCCCGACT | |
| | GGAAAGCGGGCAGTGAGCGCAACGCAATTAATGTGAGTTAGCTCACTCATTAG | |
| | GCACCCCAGGCTTTACACTTTATGCTTCCGGCTCGTATGTTGTGTGGAATTGT | |
| | GAGCGGATAACAATTTCACACAGGAAACAGCTATGACCATGATTACGCCAAGC | |
| | GCGCAATTAACCCTCACTAAAGGGAACAAAAGCTGGAGCTGCAAGCTT | |

### Cell Culture, Virus Packaging, and Transduction:

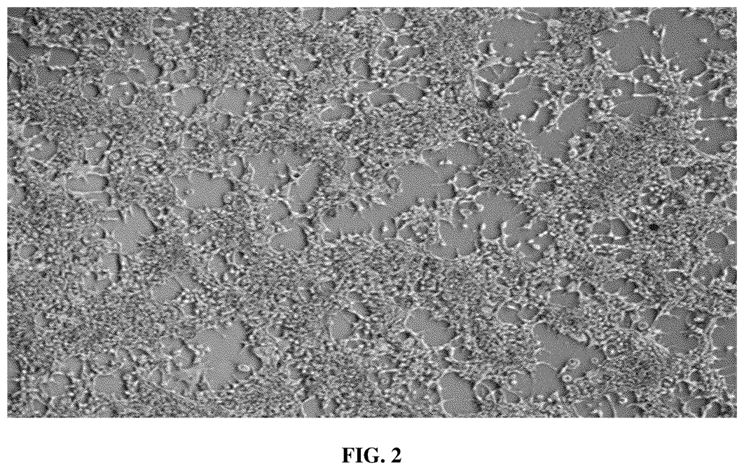

For the lentivirus packaging, the ABDNA202 construct is co-transfected with the envelope plasmid encoding VSV-G and packaging plasmids encoding Gag/Pol and Rev into HEK293T packaging cells. After 48 hours of incubation, the supernatant of the cell cultures was collected after 70 hours, centrifuged to remove cell debris and then filtered. The lentiviral particles are subsequently concentrated with PEG and cryopreserved. An aliquot of ABDNA202 lentiviral particles was used to transduce the ocular stem cells in culture. After 24 hours of viral transduction, the media of the culture was replaced with fresh one. The consumed media were collected after 72 hours of incubation and assayed for the level of secreted VEGF. As shown in FIG. 2, HEK293 cells transfected with the Lenti-Aflibercept expression construct showed the expression of a mCherry reporter at 48 hours. The mCherry positive cells could be further enriched by fluorescence-activated cell sorting (FACS).

### Expression and Secretion of Aflibercept In Vitro:

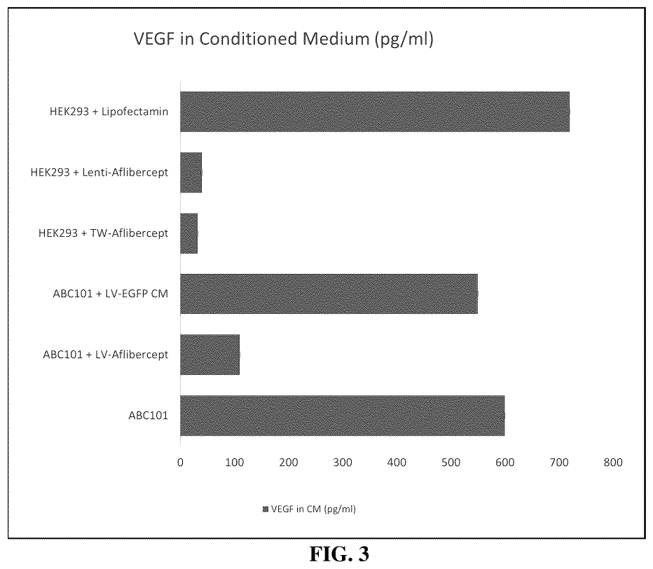

The consumed medium of lentiviral transfected ocular progenitor cells was then collected. The concentration of the free form of secreted VEGF in the conditioned medium is measured by using a Human VEGF-A ELISA kit (RayBiotech, GA). FIG. 3 illustrates the free soluble VEGF molecules in the culture supernatants of HEK293 cells and Ocular Stem Cells (ABC101), either with or without aflibercept expression constructs. Compared with the controls, such as HEK293 cells transfected with Lipofectamine, ABC101 cells alone or ABC101 cells transduced with the virus of a reporter construct LV-EGFP-mCherry, VEGF was significantly reduced in the medium supernatants of HEK293 and ABC101 culture that expressed and secreted aflibercept.

### Example 2

### Intraocular Delivery of Aflibercept

The lentiviral transfected ocular progenitor cells loaded with a drug can then be injected into the vitreous (1), the anterior chamber (2), the subretinal space (3), or the suprachoroidal space (4) (FIG. 1) of a subject's eye in need of treatment with the drug having therapeutic effects, such as aflibercept, to treat neovascular ocular conditions. The lentiviral transfected ocular progenitor cells loaded with a drug can continuously produce and release drug molecules to the surrounding ocular tissues. As disclosed herein, such sustained release of the drug molecules with very low immunogenicity can eliminate the need for frequent intravitreal injection currently being used in other methods, increase patient compliance, and thus provide much higher treatment effects in treating ocular diseases or disorders.

Unless otherwise indicated, all numbers expressing quantities of ingredients, properties such as percentages, number of cells, volumes, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

What is claimed is:

1. A method of a sustainable ocular cell-mediated intraocular delivery of a biological drug into a subject's eye in need of treatment of an ocular disease or disorder with the drug, comprising:
preparing a drug expression construct that contains the biological drug coding sequence that is linked to a promoter, wherein the biological drug is linked to a leader sequence;
introducing the drug expression construct into ocular cells in vitro to form engineered ocular cells that can express the biological drug;
formulating the engineered ocular cells to produce a therapeutic composition;
injecting the therapeutic composition comprising the engineered ocular cells into the vitreous, the anterior chamber, the subretinal space, or the suprachoroidal space of the subject's eye; and
producing and releasing drug molecules sustainably to the surrounding ocular tissues of an injection site by the engineered ocular cells that are injected into the subject's eye; and
wherein the method provides sustained therapeutic effect for treating the ocular disease or disorder.

2. The method of claim 1, wherein the biological drug is a protein, RNA, a therapeutic agent comprising a complex of multiple moieties, or a combination thereof.

3. The method of claim 2, wherein the drug is a VEGF inhibitor.

4. The method of claim 3, wherein the VEGF inhibitor is aflibercept, and wherein the amino acid sequence of aflibercept comprises SEQ ID NO: 1.

5. The method of claim 1, wherein the leader sequence is IL2 signal peptide.

6. The method of claim 1, wherein the expression construct is a viral or non-viral expression vector.

7. The method of claim 6, wherein the expression vector is a plasmid, AAV or lentiviral vector.

8. The method of claim 6, wherein the expression vector is a transgene expression vector that comprises cis-regulatory and promoter sequences that control the expression of a transgene encoding a polypeptide of a VEGF inhibitor.

9. The method of claim 8, wherein the VEGF inhibitor is aflibercept.

10. The method of claim 1, wherein the ocular cells are produced from either the direct step-wise differentiation from pluripotent stem cells or the isolation and expansion of cells from donor tissues.

11. The method of claim 1, wherein the biological drug is produced by a viral or non-viral expression construct in the engineered ocular cells; and wherein the biological drug is sustainably released or secreted from the engineered ocular cells into the subject's eye.

12. The method of claim 1, wherein the ocular cell comprises ocular stem cells.

13. The method of claim 1, wherein the ocular cell comprises an ocular cell-fate further restricted precursor cell.

14. The method of claim 13, wherein the ocular cell-fate further restricted precursor cell comprises a photoreceptor precursor cell.

15. The method of claim 13, wherein the ocular cell-fate further restricted precursor cell comprises a retinal ganglion precursor cell.

16. The method of claim 13, wherein the ocular cell-fate further restricted precursor cell comprises a retinal pigmented epithelial (RPE) cell.

17. The method of claim 13, wherein the ocular cell-fate further restricted precursor cell comprises a corneal endothelial cell.

18. The method of claim 1, wherein the ocular cell comprises the differentiated progenies of ocular stem cells.

19. A therapeutic composition for treating an ocular disease or disorder in a human subject in need thereof, comprising:
ocular cells that are introduced with a transgene expression vector in vitro, wherein the transgene expression vector comprises cis-regulatory and promoter sequences that control the expression of a transgene encoding a polypeptide of a VEGF inhibitor; and
wherein the ocular cells comprising the transgene are formulated into a suspension of cells for intraocular administration to the human subject.

20. The therapeutic composition of claim 19, wherein the transgene expression vector is an AAV or lentiviral vector.

21. The therapeutic composition of claim 19, wherein the ocular cells comprising the transgene are then cryopreserved for long-term storage.

22. The therapeutic composition of claim 21, wherein the cryopreserved ocular cells are thawed and formulated to a suspension of cells for the intraocular administration to the human subject.

23. The therapeutic composition of claim 19, wherein the ocular disease or disorder comprises neovascular AMD.

24. The therapeutic composition of claim 19, wherein the ocular cell comprises ocular stem cells.

25. The therapeutic composition of claim 19, wherein the ocular cell comprises an ocular cell-fate further restricted precursor cell.

26. The therapeutic composition of claim 25, wherein the ocular cell-fate further restricted precursor cell comprises a photoreceptor precursor cell.

27. The therapeutic composition of claim 25, wherein the ocular cell-fate further restricted precursor cell comprises a retinal ganglion precursor cell.

28. The therapeutic composition of claim 25, wherein the ocular cell-fate further restricted precursor cell comprises a retinal pigmented epithelial (RPE) cell.

29. The therapeutic composition of claim 25, wherein the ocular cell-fate further restricted precursor cell comprises a corneal endothelial cell.

30. The therapeutic composition of claim 19, wherein the ocular cell comprises a cell-fate restricted progeny of ocular stem cell.

31. A method of preparing a therapeutic composition comprising engineered ocular cells comprising:
providing an isolated ocular cell;
contacting the ocular cell with a transgene expression vector in vitro, thereby introducing the expression vector into the ocular cells to form engineered ocular cells, wherein the transgene encodes a polypeptide of a VEGF inhibitor, wherein the transgene expression vector comprises cis-regulatory and promoter sequences that control the expression of the transgene; and
formulating the engineered ocular cells into a suspension for intraocular administration.

32. The method of claim 31, wherein the transgene expression vector is AAV or lentiviral vector in vitro.

33. The method of claim 31, wherein the VEGF inhibitor is aflibercept, and wherein the amino acid sequence of aflibercept comprises SEQ ID NO: 1.

34. The method of claim 31, wherein the ocular cell comprises ocular stem cell.

35. The method of claim 31, wherein the ocular cell comprises an ocular cell-fate further restricted precursor cell.

36. The method of claim 35, wherein the ocular cell-fate further restricted precursor cell comprises a photoreceptor precursor cell.

37. The method of claim 35, wherein the ocular cell-fate further restricted precursor cell comprises a retinal ganglion precursor cell.

38. The method of claim 35, wherein the ocular cell-fate further restricted precursor cell comprises a retinal pigmented epithelial (RPE) cell.

39. The method of claim 35, wherein the ocular cell-fate further restricted precursor cell comprises a corneal endothelial cell.

40. The method of claim 31, wherein the ocular cell comprises a cell-fate restricted progeny of ocular stem cell.

41. An engineered ocular cell line, wherein cells of the engineered ocular cell line endogenously express a transgene encoding a polypeptide, wherein the polypeptide encodes a VEGF inhibitor, and wherein the cells comprise an edited genome that results in the endogenous expression of the transgene compared to a control cell line.

42. The engineered ocular cell line of claim 41, wherein the VEGF inhibitor comprises aflibercept.

43. The engineered ocular cell line of claim 41, wherein the polypeptide comprises SEQ ID NO: 1.

44. The engineered ocular cell line of claim 41, wherein the ocular cell line comprises ocular stem cells.

45. The engineered ocular cell line of claim 41, wherein the ocular cell line comprises cell-fate further restricted precursors of ocular stem cells.

46. The engineered ocular cell line of claim 45, wherein the ocular cell-fate further restricted precursors of ocular stem cells comprise a photoreceptor precursor cell.

47. The engineered ocular cell line of claim 45, wherein the ocular cell-fate further restricted precursors of ocular stem cells comprise a retinal ganglion precursor cell.

48. The engineered ocular cell line of claim 45, wherein the ocular cell-fate further restricted precursors of ocular stem cells comprise a retinal pigmented epithelial (RPE) cell.

49. The engineered ocular cell line of claim 45, wherein the ocular cell-fate further restricted precursors of ocular stem cells comprise a corneal endothelial cell.

50. A method of treating an ocular disease or disorder in a human subject in need thereof, comprising administering the engineered ocular cells of claim 31 to the human subject.

51. The method of claim 50, wherein the ocular disease or disorder is selected from the group of neovascular AMD, diabetic retinopathy, glaucoma, corneal endothelial dystrophies and inherited retinal degenerative diseases.

52. A method of treating an ocular disease or disorder in a human subject in need thereof, comprising administering the engineered ocular cells of claim 41 to the human subject.

53. The method of claim 52, wherein the ocular disease or disorder is selected from the group of neovascular AMD, diabetic retinopathy, glaucoma, corneal endothelial dystrophies and inherited retinal degenerative diseases.


SEQUENCE LISTING (10 sequences)

SEQ ID NO: 1 - protein, 430 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 2 - DNA, 1350 bp, synthetic construct
  source 1..1350: mol_type = other DNA
atgtacaggatgcaactcctgtcttgcattgcactaagtcttgcacttgtcacgaattcgagcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatcgacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaaggacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagcccc

SEQ ID NO: 3 - protein, 20 aa, unidentified
MYRMQLLSCIALILALVTNS

SEQ ID NO: 4 - protein, 36 aa, unidentified
MLPRVGCPALPLPPPPLLPLLLLLLGASGGGGGARA

SEQ ID NO: 5 - protein, 26 aa, unidentified
MNFLLSWVHWSLALLLYLHHAKWSQA

SEQ ID NO: 6 - protein, 19 aa, unidentified
MAPLRPLLILALLAWVALA

SEQ ID NO: 7 - protein, 18 aa, unidentified
MKWVTFISLLFLFSSAYS

SEQ ID NO: 8 - protein, 18 aa, unidentified
MRLLAKIICLMLWAICVA

SEQ ID NO: 9 - DNA, 232 bp, unidentified
  source 1..232: mol_type = unassigned DNA
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 10 - DNA, 9111 bp, synthetic construct
  source 1..9111: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatgtacaggatgcaactcctgtcttgcattgcactaagtcttgcacttgtcacgaattcgagcgacaccggcagacccttcgtggagatgtacagcgagatccccgagatcatccacatgaccgagggcagagagctggtgatcccctgcagagtgaccagccccaacatcaccgtgaccctgaagaagttccccctggacaccctgatccccgacggcaagagaatcatctgggacagcagaaagggcttcatcatcagcaacgccacctacaaggagatcggcctgctgacctgcgaggccaccgtgaacggccacctgtacaagaccaactacctgacccacagacagaccaacaccatcatcgacgtggtgctgagccccagccacggcatcgagctgagcgtgggcgagaagctggtgctgaactgcaccgccagaaccgagctgaacgtgggcatcgacttcaactgggagtaccccagcagcaagcaccagcacaagaagctggtgaacagagacctgaagacccagagcggcagcgagatgaagaagttcctgagcaccctgaccatcgacggcgtgaccagaagcgaccagggcctgtacacctgcgccgccagcagcggcctgatgaccaagaagaacagcaccttcgtgagagtgcacgagaaggacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagcccctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt